(19) United States
(12) Patent Application Publication
(10) Pub. No.: US 2023/0099567 A1
(43) Pub. Date: Mar. 30, 2023

(54) COMPOSITION FOR TREATMENT, PREVENTION, OR AMELIORATION OF LYMPHEDEMA

(71) Applicant: RAPPELER CO., LTD., Seoul (KR)

(72) Inventors: Jin-Mo Ku, Gyeonggi-do (KR); Sukchan Lee, Gyeonggi-do (KR); Hee Kang, Seoul (KR)

(73) Assignee: RAPPELER CO., LTD., Seoul (KR)

(21) Appl. No.: 17/964,562

(22) Filed: Oct. 12, 2022

Related U.S. Application Data
(63) Continuation of application No. PCT/KR2021/004725, filed on Apr. 14, 2021.

(30) Foreign Application Priority Data
Apr. 14, 2020 (KR) 10-2020-0045314

(51) Int. Cl. C07C 49/835; C07C 49/84; C07C 69/78; C07C 49/794; A61K 9/00; C07C 69/84; C07C 69/92; A61P 7/10

(52) U.S. Cl. CPC C07C 49/835; C07C 49/84; C07C 69/78; C07C 49/794; A61K 9/0053; C07C 69/84; C07C 69/92; A61P 7/10

(57) ABSTRACT

Provided are a novel compound exhibiting the effect of treating, preventing or ameliorating lymphedema, and a composition including the same as an active ingredient, and may be applied as a medicament product, functional food or food supplement, or cosmetics. The compound used as an active ingredient compound according to the disclosure exhibits a remarkable ameliorating effect on lymphedema in various aspects such as TNFα production inhibitory activity, metabolic stability, solubility, and blood exposure.

### CROSS-REFERENCE TO RELATED APPLICATION

This application is a continuation of International Application No. PCT/KR2021/004725 filed on Apr. 14, 2021 which claims priority under 35 U.S.C. § 119 to Korean Patent Application No. 10-2020-0045314 filed on Apr. 14, 2020, in the Korean Intellectual Property Office, the disclosure of which is incorporated by reference herein in its entirety.

### TECHNICAL FIELD

The disclosure relates to a novel compound exhibiting the effect of treating, 15 preventing, or ameliorating lymphedema, and a composition including the same as an active ingredient, and may be applied as a medicament product, functional food or food supplement, or cosmetics.

### BACKGROUND ART

Lymphedema can be largely classified as primary lymphedema caused by congenital genetic abnormalities and secondary lymphedema caused by surgery or radiation therapy. In particular, the secondary lymphedema is often developed as a sequelae after surgical treatment and/or radiation treatment for breast cancer and uterine cancer. Therefore, breast or uterine cancer patients who have undergone surgery or radiation therapy are generally considered to be potential secondary lymphedema patients.

In addition, the lymphedema can be classified as stage 0, stage 1, stage 2, and stage 3 according to the degree of the lymphedema. Stage 0 refers to the incubation stage in which there are no clinical symptoms although lymphatic movement ability is reduced, stage 1 refers to a state in which edema is noticeable and the skin is kept in for a long time when pressed, stage 2 refers to a state in which protein is accumulated in the tissue and fibrosclerosis occurs, and the skin does not enter even when pressed, and stage 3 refers to a state in which the skin is excessively inflated and the skin loses the function thereof.

In this regard, the National Cancer Information Center, hosted by the Ministry of Health and Welfare and operated by the National Cancer Center, indicates that since lymphedema is different from a symptom of swelling caused by a problem in the circulation of the lymphatic system, there is a need for an independent management and treatment method suitable for lymphedema.

In addition, the National Health Insurance Corporation says that the treatment of secondary lymphedema recommended by the World Lymphedema Association is the most effective treatment for complex lymphedema. Here, the complex lymphedema therapy refers to lymphatic massage, compression therapy, exercise and skin care. However, such a complex lymphedema therapy shows a varying efficacy depending on the mastery level of the patient, and the number and interval of the therapy, and takes a long time for the treatment. Even with this therapy, lymphedema cannot be completely cured.

As such, although lymphedema requires a therapy different from that applied to edema caused by the abnormality in the body's metabolism or kidneys, the lymphatic massage, compression therapy, exercise, skin care, etc., which are conventionally used, cannot be a fundamental and effective measure therefor.

Korean Patent Publication No. 10-1533197 (published on Jul. 2, 2015) discloses for the first time that butein or a pharmaceutically acceptable salt thereof can have a prophylactic or therapeutic effect on lymphedema or lipoedema. However, in terms of pharmacokinetics, it was confirmed that the disadvantages of butein such as solubility and blood exposure during administration of the active ingredient need to be addressed, and accordingly, in the art, there are needs for pharmaceutically, food, or cosmetically ameliorated candidate materials.

### SUMMARY

### Technical Problem

The present inventors recognized the problems of the prior art, and repeated research through numerous trials and errors in order to discover a compound having an excellent therapeutic, prophylactic or ameliorating effect on lymphedema. As a result, a compound capable of having an excellent therapeutic, prophylactic or ameliorating effect on lymphedema in various aspects such as TNFα production inhibitory activity, metabolic stability, solubility, and blood exposure was developed and completed the disclosure.

### Means of Solving the Problem

A compound according to the disclosure for achieving the objective of the disclosure is represented by Formula 1 or 2:

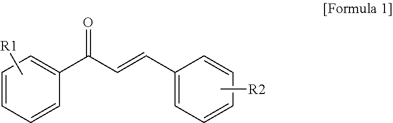

wherein, in Formula 1,

R1 and R2 may each independently be one or more selected from the group consisting of —H, —OH, —OR3, —OCOR3, —NO₂, —CN, —SH, —SR3, —COR4, —COOR4, —CONHR4, —CON(R4)₂, —NH₂, —NHR4, —N(R4)₂, —NHCOR4, —N(R5)COR4, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,

R3 may each independently be selected from the group consisting of a C₁-C₆ alkyl group, a C₃-C₁₀ aryl group,

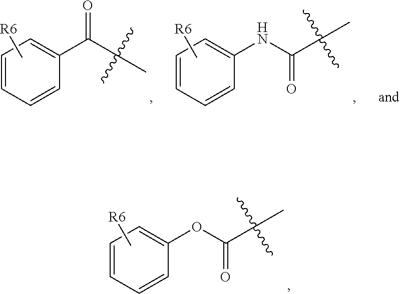

R6 may each independently be one or more selected from the group consisting of —H, —OH, —OR7, —NO₂, —CN, a C₁-C₆ alkyl group, a C₁-C₆ alkoxy group, halogen, —NH₂, —NHR7, and —N(R7)₂,

R7 may be selected from the group consisting of a C₁-C₆ alkyl group, an allyl group, and a heteroallyl group,

R4 may be selected from the group consisting of a C₁-C₆ alkyl group, an allyl group, and a heteroallyl group,

R5 may be selected from the group consisting of a C₁-C₆ alkyl group, an allyl group, and a heteroallyl group,

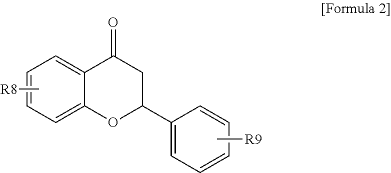

wherein, in this formula,

R8 and R9 may each independently be one or more selected from the group consisting of —H, —OH, —OR10, —NO₂, —CN, —SH, —SR10, —COR11, —COOR11, —CONHR11, —CON(R11)₂, —NH₂, —NHR11, —N(R11)₂, —NHCOR11, —N(R12)COR11, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,

R10 may each independently be selected from the group consisting of a C₁-C₆ alkyl group, a C₃-C₁₀ aryl group,

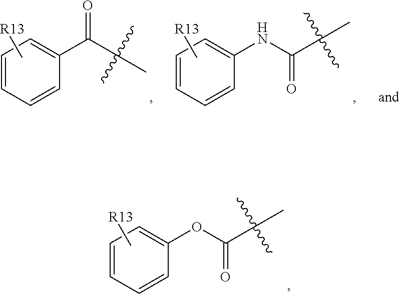

R13 may each independently be one or more selected from the group consisting of —H, —OH, —NO₂, —CN, a C₁-C₆ alkyl group, a C₁-C₆ alkoxy group, halogen, —NH₂, —NHR14, and —N(R14)₂,

R14 may be selected from the group consisting of a C₁-C₆ alkyl group, an allyl group, and a heteroallyl group,

R11 may be selected from the group consisting of a C₁-C₆ alkyl group, an allyl group, and a heteroallyl group, and

R12 may be selected from the group consisting of a C₁-C₆ alkyl group, an allyl group, and a heteroallyl group.

The term “each independently” used herein refers to that two or more substituents may be individually defined and may be different from or the same as each other. The number of substituents R1, R2, R6, R8, R9 and R13 may be one, or two or more, for example, 2, 3, 4 or 5. When the substituents R1, R2, R6, R8, R9 and R13 are two or more, the two or more substituents may be the same as or different from each other, and may be located in the ortho, meta or para position.

In an embodiment, the compound of Formula 1 may not include butein. Butein is a compound represented by the following formula, referred to herein as compound 1:

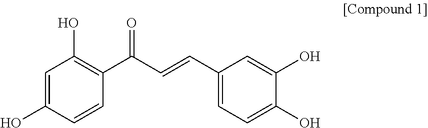 [SMILES: O=C(/C=C/c1ccc(O)c(O)c1)c1ccc(O)cc1O]

The terms “alkyl”, “alkenyl”, “alkynyl” are intended to include both straight chain (also referred to as linear) or side chain (also referred to as branched).

The term “halogen” used herein refers to fluorine, chlorine, bromine or iodine.

The term “alkoxy” used herein refers to O-alkyl.

In the term “heterocycloalkyl” used herein, “hetero” refers to a case where a hetero atom selected from oxygen, nitrogen and sulfur is included in the ring.

In an embodiment, the compound of formula 1 or 2 according to the disclosure may be selected from the group consisting of:
(E)-1-(2,4-dihydroxyphenyl)-3-(4-hydroxy-3-methoxyphenyl)prop-2-en-1-one;
(E)-1-(2,4-dihydroxyphenyl)-3-(3-hydroxy-4-methoxyphenyl)prop-2-en-1-one;
(E)-1-(2,4-dihydroxyphenyl)-3-(3-fluoro-4-hydroxyphenyl)prop-2-en-1-one;
(E)-1-(2,4-dihydroxyphenyl)-3-(4-fluoro-3-hydroxyphenyl)prop-2-en-1-one;
(E)-1-(2,4-dihydroxyphenyl)-3-(4-hydroxyphenyl)prop-2-en-1-one;
(E)-1-(2,4-dihydroxyphenyl)-3-(3-hydroxyphenyl)prop-2-en-1-one;
(E)-3-(3,4-dihydroxyphenyl)-1-(2-fluoro-4-hydroxyphenyl)prop-2-en-1-one;
(E)-3-(3,4-dihydroxyphenyl)-1-(4-hydroxy-2-methylphenyl)prop-2-en-1-one;
(E)-3-(3,4-dihydroxyphenyl)-1-(4-hydroxy-2-methoxyphenyl)prop-2-en-1-one;
(E)-3-(3,4-dihydroxyphenyl)-1-(4-hydroxy-2-isopropoxyphenyl)prop-2-en-1-one;
(E)-3-(3,4-dihydroxyphenyl)-1-(2,4-dimethoxyphenyl)prop-2-en-1-one;
(E)-3-(3,4-dihydroxyphenyl)-1-(p-tolyl)prop-2-en-1-one;
(E)-1-(4-chlorophenyl)-3-(3,4-dihydroxyphenyl)prop-2-en-1-one;
(E)-4-(3-(2,4-diacetoxyphenyl)-3-oxoprop-1-en-1-yl)-1,2-phenylene diacetate;
(E)-4-(3-(2,4-bis(benzoyloxy)phenyl)-3-oxoprop-1-en-1-yl)-1,2-phenylene dibenzoate;
(E)-2-(3-(3,4-dihydroxyphenyl)acryloyl)-5-hydroxyphenyl benzoate;
(E)-2-(3-(3,4-dihydroxyphenyl)acryloyl)-5-hydroxyphenyl 4-methylbenzoate;
(E)-2-(3-(3,4-dihydroxyphenyl)acryloyl)-5-hydroxyphenyl 4-methoxybenzoate, or
(E)-2-(3-(3,4-dihydroxyphenyl)acryloyl)-5-hydroxyphenyl 4-chlorobenzoate.

The terms “pharmaceutically acceptable salt” and “food-acceptable salt” used herein are those that can be prepared by a method conventional in the art. Examples thereof are: a pharmaceutically or food-acceptable salt thereof with an inorganic acid, such as hydrochloric acid, hydrogen bromide, sulfuric acid, sodium hydrogen sulfate, phosphoric acid, or carbonic acid, or an organic acid, such as formic acid, acetic acid, oxalic acid, benzoic acid, citric acid, tartaric acid, gluconic acid, gestisic acid, fumaric acid, lactobionic acid, salicylic acid, or acetylsalicylic acid (Aspirin); a metal salt thereof obtained by reacting with alkali metal ions of sodium and potassium; or another type of pharmaceutically or food-acceptable salt thereof obtained by reacting with ammonium ions, but the disclosure is not limited thereto.

The disclosure provides a composition for treating or preventing lymphedema including a compound represented by Formula 1 or 2 or a pharmaceutically acceptable salt thereof as an active ingredient.

The term “treatment” used herein refers to any action in which symptoms of lymphedema are ameliorated or cured by administration of the composition according to the disclosure.

The term “prevention” used herein refers to any action in which symptoms of lymphedema are suppressed or delayed by administration of the composition according to the disclosure.

It should be understood that the lymphedema includes all symptoms, diseases, conditions, etc. which are additionally developed by the progression of lymphedema or are developed accompanying lymphedema, or in which the severity of lymphedema is increased.

The pharmaceutical composition according to the disclosure may include the compound represented by Formula 1 or 2 or a pharmaceutically acceptable salt thereof alone, or may additionally include one or more pharmaceutically acceptable carriers, excipients or diluents.

The pharmaceutically acceptable carrier may further include, for example, a carrier for oral administration or a carrier for parenteral administration. Carriers for oral administration may include lactose, starch, cellulose derivatives, magnesium stearate, stearic acid, and the like. In addition, the carrier for parenteral administration may include water, a suitable oil, saline, aqueous glucose and glycol, and the like, and may further include a stabilizer and a preservative. Suitable stabilizers may include antioxidants such as sodium hydrogen sulfite, sodium sulfite or ascorbic acid. Suitable preservatives may include benzalkonium chloride, methyl- or propyl-paraben and chlorobutanol. As other pharmaceutically acceptable carriers, those may be referred to disclosed in the following document (Remington's Pharmaceutical Sciences, 19th ed., Mack Publishing Company, Easton, Pa., 1995).

The pharmaceutical composition of the disclosure may be administered to mammals including humans by any method. For example, oral or parenteral administration may be performed. Parenteral administration methods may include, but are not limited to, intravenous administration, intramuscular administration, intraarterial administration, intramedullary administration, intrathecal administration, intracardiac administration, transdermal administration, subcutaneous administration, intraperitoneal administration, intranasal administration, enteral administration, topical administration, or sublingual or rectal administration. For example, the pharmaceutical composition of the disclosure may be prepared in the form of an injectable formulation, which can be administered by lightly pricking the skin with a 30-gauge thin injection needle, or by directly applying the same on the skin.

The pharmaceutical composition of the disclosure may be formulated as a formulation for oral administration or parenteral administration according to the administration route as described above.

In the case of a formulation for oral administration, the composition of the disclosure may be formulated in the form of a powder, granule, tablet, pill, dragee, capsule, liquid, gel, syrup, slurry, suspension, etc. using methods known in the art. For example, oral preparations can be obtained by mixing an active ingredient with a solid excipient, pulverizing the same, adding a suitable adjuvant thereto, and processing the same into a granule mixture to obtain tablets or dragees. Examples of suitable excipients include: sugars including lactose, dextrose, sucrose, sorbitol, mannitol, xylitol, erythritol, and maltitol; starches, including corn starch, wheat starch, rice starch, and potato starch; celluloses including cellulose, methyl cellulose, sodium carboxymethylcellulose and hydroxypropylmethyl-cellulose, and the like; and fillers such as gelatin, polyvinylpyrrolidone, and the like. In addition, cross-linked polyvinylpyrrolidone, agar, alginic acid or sodium alginate may be added as a disintegrant if necessary. Furthermore, the composition of the disclosure may further include an anti-aggregating agent, a lubricant, a wetting agent, a flavoring agent, an emulsifying agent, an antiseptic agent, and the like.

Formulations for parenteral administration may be formulated in the form of injections, creams, lotions, external ointments, oils, moisturizers, gels, aerosols, and nasal inhalants, by methods known in the art. These formulations are described in the document of Remington's Pharmaceutical Science, 15th Edition, 1975. Mack Publishing Company, Easton, Pa. 18042, Chapter 87: Blaug, Seymour, which is a recipe commonly known to all pharmaceutical chemistry.

The total effective amount of the pharmaceutical composition of the disclosure may be administered to a patient as a single dose, and may be administered according to a fractionated treatment protocol in which multiple doses are administered for a long period of time. The amount of the active ingredient of the pharmaceutical composition of the disclosure may vary depending on the symptoms of the disease. Preferably, the preferable total dose of the composition of the disclosure may be from about 0.01 μg to 1,000 mg, the most preferably, from 0.1 μg to 100 mg, per kg of patient body weight per day. However, regarding the dosage of the pharmaceutical composition of the disclosure, those of ordinary skill in the art may determine an appropriate effective dosage in consideration of not only the route of administration and the number of treatments, but also various factors such as the age, weight, health status, sex, severity of the disease, diet and excretion rate of the patient. The pharmaceutical composition according to the disclosure is not particularly limited to the formulation, the administration route, and the administration method as long as the effect of the disclosure is exhibited.

In addition, the pharmaceutical composition of the disclosure may be administered as an individual therapeutic agent or in combination with other therapeutic agents. When administered in combination with other therapeutic agents, the composition of the disclosure and the other therapeutic agents may be administered simultaneously, separately, or sequentially. The other therapeutic agents may be any material that is already known to have the effect of treating or ameliorating lymphedema. The other therapeutic agents may include all surgical procedures, surgery, and the like other than drug therapy.

When the pharmaceutical composition of the disclosure is administered in combination with other therapeutic agents, the composition of the disclosure and the other therapeutic agents may be formulated separately in separate containers, or may be formulated together in the same container.

Another aspect of the disclosure provides a method of treating or preventing lymphedema comprising administering the composition of the disclosure to a subject.

In the method for treating or preventing lymphedema according to the disclosure, each term has the same meaning as described above in connection with the composition for treating or preventing lymphedema unless specified otherwise.

The term “subject” includes any human or non-human animal. The term “non-human animal” may be a vertebrate, such as non-human primates, sheep, dogs, and rodents, such as mice, rats and guinea pigs. The subject may be a human. The term “subject” is used interchangeably herein with “individual” and “patient.”

In the method for treating or preventing lymphedema according to the disclosure, the composition of the disclosure may be administered to a subject simultaneously, sequentially, or separately with other therapeutic agents. The “simultaneous” administration refers to that the composition of the disclosure and another therapeutic agent are administered at one time through the same injection method. The “sequential” administration refers to administering the composition of the disclosure and another therapeutic agent by using separate injection methods, wherein the administering is a relatively continuous administration, and the time consumed in the administration intervals is allowed to be as small as possible. The “separate” administration refers to administration of the composition of the disclosure and another therapeutic agent at regular time intervals. The administration method of the composition of the disclosure and other therapeutic agents may be appropriately selected by those of ordinary skill in the art in consideration of the therapeutic efficacy and side effects of the patient.

Another aspect of the disclosure provides a food composition for ameliorating or preventing lymphedema, including the compound represented by Formula 1 or 2 or a food-acceptable salt thereof as an active ingredient.

In the food composition according to the disclosure, each term has the same meaning as described above in connection with the pharmaceutical composition for treating or preventing lymphedema unless specified otherwise.

The term “amelioration” refers to any action in which the degree of lymphedema or symptoms associated therewith is reduced or ameliorated, or the progress thereof is delayed by administration of the composition according to the disclosure.

The food may be a health functional food. The term “health functional food” refers to food manufactured and processed in the form of tablets, capsules, powders, granules, liquids, pills, etc. using raw materials or ingredients useful in the human body.

The term “functional” refers to obtaining of useful effects for health purposes such as regulating nutrients or physiological actions with respect to the structure and function of the human body.

The food composition according to the disclosure can be prepared by a method commonly used in the art, and during the preparation, raw materials and components commonly added in the art may be added thereto. In addition, unlike general drugs, due to the use of food as a raw material, any side effect that may occur when the drug is taken for a long time, may not occur. Also, the portability is excellent. Accordingly, the food composition of the disclosure may be taken as a supplement for the enhancement or improvement of the therapeutic effect of lymphedema.

The amount of the compound or salt thereof included as an active ingredient in the food composition according to the disclosure may be appropriately determined depending on the purpose of use (prevention, amelioration, or therapeutic treatment).

In general, when preparing food, the compound or salt thereof according to the disclosure may be included in an amount of 0.001 wt % to 20 wt %, 0.001 wt % to 15 wt %, or 0.001 wt % to 10 wt % of the composition. In the case of health drinks, the compound or salt thereof according to the disclosure may be included in an amount of 0.01 g to 2 g, 0.02 g to 2 g, or 0.3 g to 1 g may be added, based on 100 mL. However, in the case of health and hygiene purposes or long-term intake for health control purposes, the amount thereof may be outside these amount ranges.

The amount of the compound or salt thereof according to the disclosure added to the food composition in the process of preparing the food composition may be appropriately increased or decreased according to purpose.

The food composition of the disclosure may further include additional ingredients in addition to the compound or salt thereof to enhance efficacy.

The food composition may be in any one formulation selected from the group consisting of pills, tablets, granules, powders, capsules, and liquid solutions.

In addition, the type of the food is not particularly limited. Examples of foods to which the material can be added include meat, sausage, bread, chocolate, candy, snacks, confectionery, pizza, instant noodles, other noodles, gums, dairy products including ice cream, various soups, beverages, tea, drinks, alcoholic beverages, vitamin complexes, and the like, and includes all foods in the ordinary sense.

Like conventional food, the food composition of the disclosure may include various flavoring agents or natural carbohydrates as additional ingredients. The natural carbohydrates include monosaccharides such as glucose and fructose, disaccharides such as maltose and sucrose, polysaccharides such as dextrin and cyclodextrin, and sugar alcohols such as xylitol, sorbitol, and erythritol. As the sweetener, natural sweeteners such as taumatine and stevia extract, synthetic sweeteners such as saccharin and aspartame, and the like may be used.

When the food composition of the disclosure is a beverage composition, there is no particular limitation on the liquid component except for containing the compound or salt thereof in the indicated ratio as an essential component, and like a conventional beverage, various flavoring agents or natural carbohydrates may be added as an additional component.

Another aspect of the disclosure provides a cosmetic composition for ameliorating or preventing lymphedema, including the compound represented by Formula 1 or 2 or a cosmetically acceptable salt thereof as an active ingredient.

In the cosmetic composition according to the disclosure, each term has the same meaning as described above in the pharmaceutical composition or food composition unless specified otherwise.

The cosmetic composition of the disclosure may be applied alone, and may be applied simultaneously with the pharmaceutical composition and/or food composition of the disclosure, and the effect of treating, preventing or ameliorating lymphedema may be enhanced.

The cosmetic composition according to the disclosure may be prepared by a method commonly used in the art, and during the preparation, raw materials and components commonly added in the art may be added thereto.

The amount of the compound or salt thereof included as an active ingredient in the cosmetic composition according to the disclosure may be appropriately determined in consideration of various factors such as the purpose of use (prevention, amelioration, or therapeutic treatment), the period of use, the type of formulation, the route of administration, and the skin condition of the subject. For example, the amount of the compound or salt thereof may be 0.001 wt % to 10 wt %, for example, 0.01 wt % to 5 wt %, based on the total weight of the cosmetic composition. According to the disclosure, the cosmetic composition may be prepared in the form of a general emulsified formulation and a solubilized formulation using a conventionally known manufacturing method. In this regard, the cosmetic composition of the disclosure may be prepared in various formulations such as patches, ointments, gels for skin adhesion, creams, packs, lotions, essences, sprays, masks, foundations, makeup bases, detergents, water (W) type, oil (O) type, silicone (S) type, oil-in-water (O/W) type, water-in-oil (W/O) type, water-in-silicone (W/S) type, silicone in water (S/W) type, solid, liquid, and the like.

The cosmetic composition according to the disclosure may further include additional ingredients in addition to the compound or salt thereof to enhance efficacy. For example, such additional ingredients are not limited as long as they do not counteract or reduce the efficacy of the compound or salt thereof according to the disclosure. Optionally, adjuvants, carriers, etc., which are commonly used in the cosmetic field, may be additionally included. Optionally, ingredients which are typically used to add or enhance cosmetic functions may also be included. For example, one or more aqueous additives selected from stabilizers, emulsifiers, thickeners, humectants, liquid crystal film strengthening agents, pH regulators, antibacterial agents, water-soluble polymers, film agents, metal ion blockers, amino acids, organic amines, polymer emulsions, pH regulators, skin nutrients, antioxidants, antioxidant auxiliaries, preservatives, fragrances, and the like, and at least one oily additive selected from oils and fats, waxes, hydrocarbon oil, higher fatty acid oil, higher alcohol, synthetic ester oil, silicone oil, and the like, may be added

### Advantages Effects of Disclosure

The composition according to the disclosure can significantly reduce the size of lymphedema, and can achieve the effect of reducing the size of lymphedema in a shorter time than the conventionally used therapy for treating or ameliorating lymphedema. In addition, the composition according to the disclosure exhibits an effect of inhibiting, blocking, or preventing the new generation or expansion of lymphedema.

Accordingly, the composition according to the disclosure can exhibit excellent therapeutic and ameliorating effects to a desired degree within a short period of time for a subject already suffering from lymphedema. In addition, in regard to subjects with the potential to have lymphedema (e.g., patients who have undergone cancer surgery and/or radiation therapy), the effect of preventing lymphedema in advance can be obtained, and accordingly, the incidence rate of lymphedema significantly is lowered.

Such a composition of the disclosure can be provided in the form of a pharmaceutical product, and alternatively, may be provided in the form of a functional food or food supplement, and alternatively, may be provided in the form of a cosmetic.

### BRIEF DESCRIPTION OF DRAWINGS

FIG. 1 is a graph showing the level of LPS-induced TNFα production in mouse peritoneal macrophages.

FIG. 2A shows images showing the change in the size of lymphedema on the 7^{th} day after surgery in the normal group, the control group, and the compound-treated group of the disclosure with respect to lymphedema induced in mice, and FIG. 2B is a graph in which the volumes of lymphedema in the respective groups are compared.

FIG. 3A shows an H&E image of the right hind leg of a mouse taken through immunofluorescence analysis in a normal group, a control group, and a group treated with the compound of the disclosure.

FIG. 3B is a graph in which mRNA expression of TNFα in a lymphedema tissue of the right hind limb of a mouse can be compared.

FIG. 4A shows an H&E image of the right hind leg of a mouse taken through immunofluorescence analysis in a normal group, a control group, and a group treated with the compound of the disclosure.

FIG. 4B shows image in which the distribution of PPARγ, which is a major regulator of lipid production in the epidermal layer of lymphedema tissue, can be identified.

FIG. 4C shows a western blot image in which the expression of PPARγ and a target protein Fabp4 thereof in lymphedema tissue can be identified.

FIG. 4D is a graph in which the expression of PPARγ and a target protein Fabp4 thereof in lymphedema tissue can be compared.

### DETAILED DESCRIPTION

Hereinafter, the disclosure will be described in more detail using Examples. However, these examples are for illustrative purposes only, and the scope of the disclosure is not limited by these examples. In addition, those of ordinary skill in the art will be able to make various changes and modifications to the disclosure within the scope that does not impair the spirit of the disclosure. Terms not specifically defined in this specification should be understood to have meanings commonly used in the technical field to which the disclosure pertains.

All animal tests in the following examples were performed after obtaining approvals by the Institutional Animal Care and Use Committee (IACUC) of Sungkyunkwan University School of Medicine (Approval No.: SKKUIACUC-20150037). All methods were performed according to the approved guidelines. A murine model for acquired lymphedema was surgically induced in the legs of male ICR mice using an established protocol. Briefly, major lymphatic sites including inguinal lymph nodes, popliteal lymph nodes, deep inguinal lymph nodes and femoral lymphatic vessels were removed, and the lower right leg of the mouse was limitedly cauterized under anesthesia.

Compounds of Formula 1 or 2 were prepared according to Scheme 1 or Scheme 2. For example, compounds 1, 7a-m, 8a-b, and 9 described below were prepared according to scheme 1, and compounds 14a-d was prepared according to scheme 2.

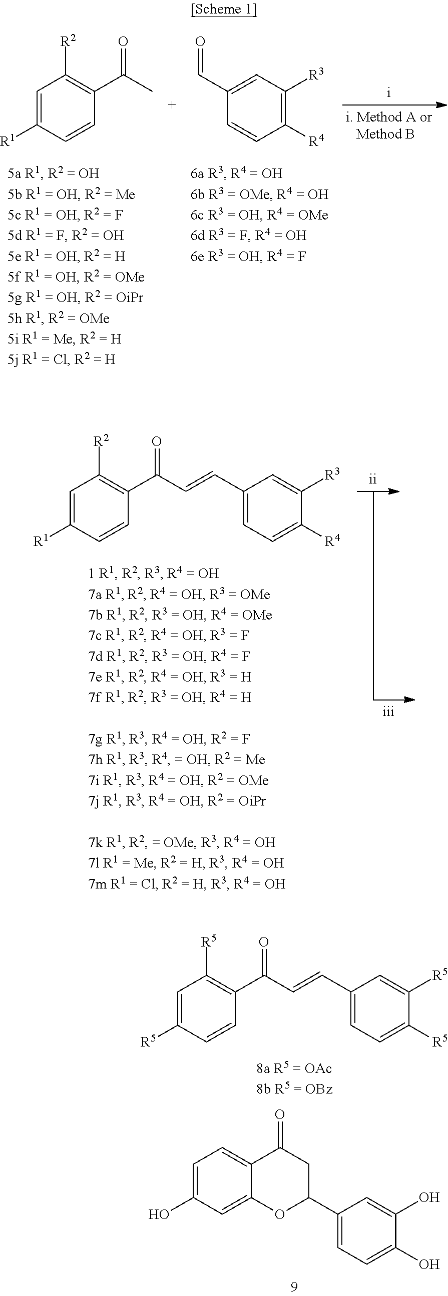

Reagents and conditions in Scheme 1 are as follows:

(i) Preparation A: 60% KOH(aq), ethanol, room temperature, 6 h, 60-72%; Preparation B: 10% CuBr₂, ethyl acetate, 80° C., 8 h, 42-60%;

(ii) acetic anhydride, reflux, overnight, 93% or benzoyl chloride, TEA, THF, 6 h, 65%; and

(iii) KOH(s), methanol, reflux, 4 h, 60%.

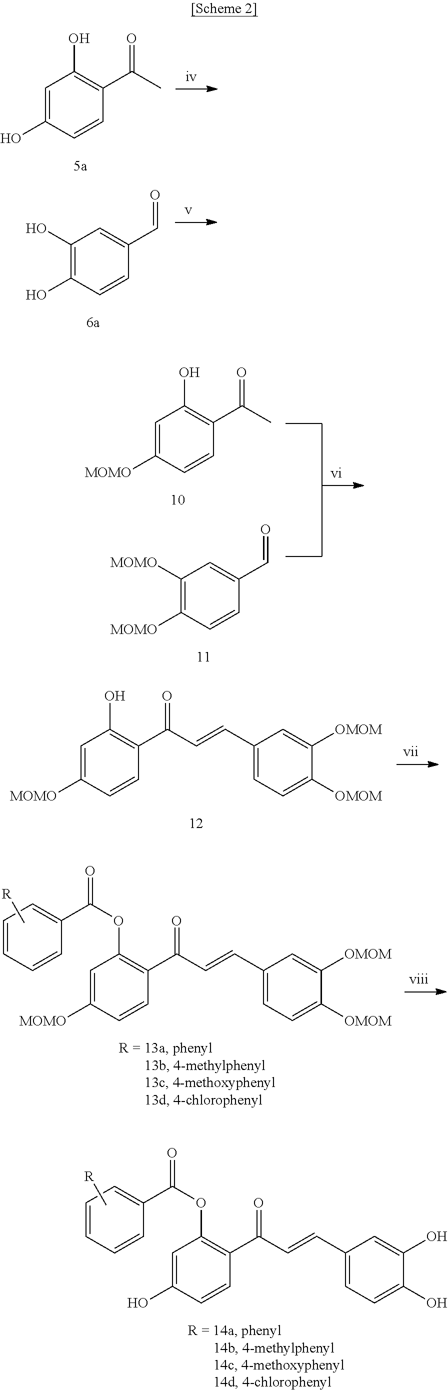

Reagents and conditions in Scheme 2 are as follows:

(iv) methoxymethyl chloride, K₂CO₃, actone, room temperature, 4 h, 61%;

(v) methoxymethyl chloride, K₂CO₃, acetone, reflux, 2.5 h, 70%;

(vi) 60% KOH(aq), ethanol, room temperature, 6 h, 70%;

(vii) acyl chloride, TEA, DMF, room temperature, 2-3 h, 60%-73%; and

(viii) 6N HCl (aq), ethanol, room temperature, 4 h, 60%-75%.

Hereinafter, specific preparation steps for the preparation of the compound according to the disclosure and representative examples corresponding thereto are described together. In the case of compounds having different substituents, these compounds were actually prepared through similar steps, but not all preparation examples are specified in the present specification. Those of ordinary skill in the art will be able to easily prepare compounds of Formula 1 or 2 having different substituents with reference to the following representative examples.

### Preparation A

### Compound 1: (E)-1-(2,4-dihydroxyphenyl)-3-(3,4-dihydroxyphenyl)prop-2-en-1-one

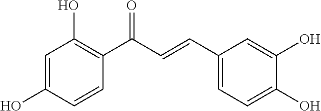 [SMILES: O=C(/C=C/c1ccc(O)c(O)c1)c1ccc(O)cc1O]

Compound 1 refers to butein.

3,4-dihydroxybenzaldehyde (compound 6a, 1.0 g, 7.2 mmol) and 1-(2,4-dihydroxyphenyl)ethan-1-one (compound 5a, 1.1 g, 7.2 mmol) were dissolved in ethanol (10 mL). 60% KOH (aq) (1 mL) was added and the mixture was left at room temperature for 6 h. The solvent was removed therefrom under reduced pressure, and the residue was extracted three times with 30 ml of diethyl ether. The diethyl ether layer was washed three times with water (30 ml×3) and dried using anhydrous MgSO₄.

After removing the solvent, compound 1 was obtained by silica gel column chromatography (silica gel: n-hexane-ethyl acetate=1:1) (yellow solid, 42% yield). ¹H NMR, ¹³C NMR, LRMS, HRMS, and M.P data were measured as follows:

¹H NMR (MeOD, 400 MHz) b 7.95 (1H, d, J=8.8 Hz), 7.73 (1H, d, J=15.6 Hz), 7.54 (1H, d, J=15.6 Hz), 7.20 (1H, d, J=2.0 Hz), 7.12 (1H, dd, J=8.4, 2.4 Hz), 6.83 (1H, d, J=8.4 Hz), 6.43 (1H, dd, J=8.8, 2.4 Hz), 6.31 (1H, d, J=2.4 Hz); ¹³C NMR (100 MHz, MeOD) b 193.50, 167.52, 166.37, 149.95, 146.86, 146.11, 133.30, 128.45, 123.64, 118.31, 116.63, 115.84, 114.74, 109.17, 103.85; LRMS (ESI) calcd. for C₁₅H₁₃O₅ [M+H]⁺: 273.08, found: 273.10; HRMS (ESI) calcd. for C₁₅H₁₃O₅ [M+H]⁺: 273.0685, found: 273.0754; M.P=214.7° C.

### Preparation B

1-(4-hydroxy-2-methylphenyl)ethan-1-one (compound 5b, 150 mg, 1.0 mmol), 2,4-dihydroxybenzaldehyde (compound 6a, 138 mg, 1.0 mmol) and CuBr₂ (22 mg, 0.1 mmol) were dissolved in 5 ml ethyl acetate at room temperature in a pressurized tube, and the reaction mixture was stirred at 80° C. After 12 h, the mixture was cooled to room temperature and filtered through Celite.

The organic layer was concentrated in a vacuum condition, and compound 7h was obtained by silica gel column chromatography (silica gel:n-hexane-ethylacetate=1:1) (yellow solid, 66% yield).

### Compound 7a: (E)-1-(2,4-dihydroxyphenyl)-3-(4-hydroxy-3-methoxyphenyl)prop-2-en-1-one

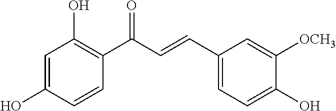 [SMILES: COc1cc(/C=C/C(=O)c2ccc(O)cc2O)ccc1O]

Compound 7a was isolated via Preparation A (yellow solid, 46% yield), and ¹H NMR, ¹³C NMR, LRMS, HRMS and MP data were measured as follows:

¹H NMR (MeOD, 400 MHz) b 8.06 (1H, d, J=8.8 Hz), 7.69 (1H, d, J=15.2 Hz), 7.55 (1H, d, J=15.2 Hz), 7.27 (1H, d, J=2.0 Hz), 7.12 (1H, dd, J=8.4, 2.0 Hz), 6.75 (1H, d, J=8.0 Hz), 6.31 (1H, dd, J=8.8, 2.4 Hz), 6.19 (1H, d, J=2.4 Hz) 3.84 (3H, s); ¹³C NMR (100 MHz, MeOD) b 193.4, 167.5, 166.4, 151.7, 148.0, 145.5, 133.4, 129.5, 123.5, 119.2, 115.2, 114.7, 112.5, 109.2, 103.8, 56.56; LRMS (ESI) calcd. for C₁₆H₁₅O₅ [M+H]⁺: 287.09, found: 287.10; HRMS (ESI) calcd. for C₁₆H₁₅O₅ [M+H]⁺: 287.0914, found: 287.0908; M.P=198.1° C.

### Compound 7b: (E)-1-(2,4-dihydroxyphenyl)-3-(3-hydroxy-4-methoxyphenyl)prop-2-en-1-one

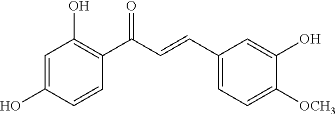 [SMILES: COc1ccc(/C=C/C(=O)c2ccc(O)cc2O)cc1O]

Compound 7b was isolated via Preparation A (yellow solid, 48% yield), and ¹H NMR, ¹³C NMR, LRMS, HRMS and MP data were measured as follows:

¹H NMR (400 MHz, MeOD) b 7.96 (1H, d, J=8.0 Hz), 7.74 (1H, d, J=15.6 Hz), 7.59 (1H, d, J=15.2 Hz), 7.25 (1H, d, J=2.4 Hz), 7.20 (1H, dd, J=8.4, 2.0 Hz), 6.98 (1H, d, J=8.4 Hz), 6.43 (1H, dd, J=8.8, 2.4 Hz), 6.31 (1H, d, J=2.4 Hz) 3.92 (3H, s); ¹³C NMR (100 MHz, MeOD) b 193.4, 167.5, 166.4, 151.7, 148.0, 145.5, 133.4, 129.5, 123.5, 119.2, 115.2, 114.7, 112.5, 109.2, 103.8, 56.39; LRMS (ESI) calcd. for C₁₆H₁₅O₅ [M+H]⁺: 287.09, found: 287.10; HRMS (ESI) calcd. for C₁₆H₁₅O₅ [M+H]⁺: 287.0914, found: 287.0908; M.P=206.4° C.

### Compound 7c: (E)-1-(2,4-dihydroxyphenyl)-3-(3-fluoro-4-hydroxyphenyl)prop-2-en-1-one

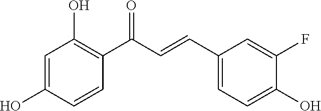 [SMILES: O=C(/C=C/c1ccc(O)c(F)c1)c1ccc(O)cc1O]

Compound 7c was isolated via Preparation A (yellow solid, 53% yield), and ¹H NMR, ¹³C NMR, LRMS, HRMS and MP data were measured as follows:

¹H NMR (400 MHz, MeOD δ 7.99 (1H, d, J=8.8 Hz), 7.75 (1H, d, J=15.2 Hz), 7.65 (1H, d, J=15.6 Hz), 7.57 (1H, dd, J=12.4, 2.0 Hz), 7.39 (1H, dd, J=8.4, 1.6 Hz), 6.97 (1H, t, J=8.8 Hz), 6.43 (1H, dd, J=8.8, 2.4 Hz), 6.30 (1H, d, J=2.4 Hz); ¹³C NMR (100 MHz, MeOD) δ 193.2, 167.5, 166.5, 154.2, 151.9, 149.1, 144.1, 133.5, 127.4, 119.9, 118.9, 116.5, 114.6, 109.2, 103.8; LRMS (ESI) calcd. for C₁₅H₁₂O₄F [M+H]⁺: 275.07, found: 275.10; HRMS (ESI) calcd. for C₁₅H₁₂O₄F [M+H]⁺: 275.0714, found: 275.0709; M.P=210.5° C.

### Compound 7d: (E)-1-(2,4-dihydroxyphenyl)-3-(4-fluoro-3-hydroxyphenyl)prop-2-en-1-one

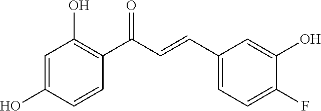 [SMILES: O=C(/C=C/c1ccc(F)c(O)c1)c1ccc(O)cc1O]

Compound 7d was isolated via Preparation A (yellow solid, 52% yield) and ¹H NMR, ¹³C NMR, LRMS, HRMS and MP data were measured as follows:

¹H NMR (400 MHz, MeOD) δ 7.98 (1H, d, J=8.8 Hz), 7.74 (1H, d, J=15.6 Hz), 7.67 (1H, d, J=15.6 Hz), 7.32 (1H, dd, J=8.4, 2.0 Hz), 7.25-7.21 (1H, m), 7.15-7.10 (1H, m), 6.44 (1H, dd, J=8.8, 2.4 Hz), 6.31 (1H, d, J=2.4 Hz); ¹³C NMR (100 MHz, MeOD) δ 193.2, 167.6, 166.7, 155.7, 146.7, 144.3, 133.5, 133.2, 122.0, 121.4, 118.5, 117.4, 114.6, 109.3, 103.8; LRMS (ESI) calcd. for C₁₅H₁₂O₄F [M+H]⁺: 275.07, found: 275.10; HRMS (ESI) calcd. for C₁₅H₁₂O₄F [M+H]⁺: 275.0714, found: 275.0709; M.P=252.4° C.

### Compound 7e: (E)-1-(2,4-dihydroxyphenyl)-3-(4-hydroxyphenyl)prop-2-en-1-one

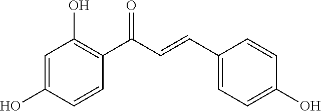 [SMILES: O=C(/C=C/c1ccc(O)cc1)c1ccc(O)cc1O]

Compound 7e was isolated via Preparation A (yellow solid, 43% yield), and ¹H NMR, ¹³C NMR, LRMS, HRMS and MP data were measured as follows:

¹H NMR (400 MHz, MeOD) δ 7.98 (1H, d, J=9.2 Hz), 7.80 (1H, d, J=15.2 Hz), 7.64-7.60 (3H, m), 6.86 (2H, d, J=8.0 Hz), 6.43 (1H, dd, J=8.8, 2.4 Hz), 6.31 (1H, d, J=2.4 Hz); ¹³C NMR (100 MHz, MeOD) δ 193.5, 167.5, 166.3, 161.5, 145.6, 133.3, 131.8, 127.8, 118.5, 116.9, 114.7, 109.1, 103.8; LRMS (ESI) calcd. for C₁₅H₁₃O₄ [M+H]⁺: 257.08, found: 257.10; HRMS (ESI) calcd. for C₁₅H₁₃O₄ [M+H]⁺: 257.0801, found: 257.0801; M.P=205.7° C.

### Compound 7f: (E)-1-(2,4-dihydroxyphenyl)-3-(3-hydroxyphenyl)prop-2-en-1-one

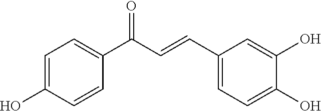 [SMILES: O=C(/C=C/c1ccc(O)c(O)c1)c1ccc(O)cc1]

Compound 7f was isolated via Preparation A (yellow solid, 52% yield), and 1H NMR, ¹³C NMR, LRMS, HRMS and MP data were measured as follows:

¹H NMR (400 MHz, MeOD) δ 8.00 (2H, d, J=8.8 Hz), 7.66 (1H, d, J=15.6 Hz), 7.51 (1H, d, J=15.2 Hz), 7.19 (1H, d, J=2.4 Hz), 7.11 (1H, dd, J=8.0, 2.0 Hz), 6.91 (2H, d, J=8.8 Hz), 6.83 (1H, d, J=8.0 Hz); ¹³C NMR (100 MHz, MeOD) δ 191.0, 163.7, 149.8, 146.2, 132.2, 131.2, 128.4, 123.4, 119.5, 116.6, 116.4, 115.6; LRMS (ESI) calcd. for C₁₅H₁₃O₄ [M+H]⁺: 257.08, found: 257.10; HRMS (ESI) calcd. for C₁₅H₁₃O₄ [M+H]⁺: 257.0808, found: 257.0803; M.P=208.0° C.

### Compound 7g: (E)-3-(3,4-dihydroxyphenyl)-1-(2-fluoro-4-hydroxyphenyl)prop-2-en-1-one

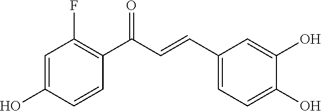 [SMILES: O=C(/C=C/c1ccc(O)c(O)c1)c1ccc(O)cc1F]

Compound 7g was isolated via Preparation A (yellow solid, 53% yield) and ¹H NMR, ¹³C NMR, LRMS, HRMS and MP data were measured as follows:

¹H NMR (400 MHz, MeOD) δ 7.75 (1H, t, J=8.8 Hz), 7.62 (1H, dd, J=15.6, 2.0 Hz), 7.28 (1H, dd, J=15.2, 2.4 Hz), 7.14 (1H, d, J=2.0 Hz), 7.19 (1H, d, J=2.4 Hz), 7.04 (1H, dd, J=8.4, 2.0 Hz), 6.82 (1H, d, J=8.0 Hz), 6.72 (1H, dd, J=8.8, 2.4 Hz), 6.61 (1H, dd, J=13.2, 2.4 Hz); ¹³C NMR (100 MHz, MeOD) δ 189.5, 165.0, 150.0, 146.8, 133.6, 128.2, 123.6, 123.2, 119.6, 116.6, 115.4, 113.1, 104.2, 103.9; LRMS (ESI) calcd. for C₁₅H₁₂O₄F[M+H]⁺: 275.08, found: 275.10; HRMS (ESI) calcd. for C₁₅H₁₂O₄F [M+H]⁺: 275.0714, found: 275.0708; M.P=207.8° C.

### Compound 7h: (E)-3-(3,4-dihydroxyphenyl)-1-(4-hydroxy-2-methylphenyl)prop-2-en-1-one

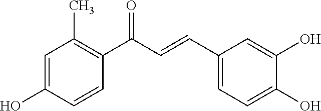 [SMILES: Cc1cc(O)ccc1C(=O)/C=C/c1ccc(O)c(O)c1]

Compound 7h was isolated via Preparation B (yellow solid, 66% yield) and ¹H NMR, ¹³C NMR, LRMS, HRMS and MP data were measured as follows:

¹H NMR (400 MHz, MeOD) δ 7.54 (1H, d, J=9.2 Hz), 7.41 (1H, d, J=15.6 Hz), 7.12 (1H, d, J=2.4 Hz), 7.09 (1H, d, J=16.0 Hz), 7.02 (1H, dd, J=8.0, 2.0 Hz), 6.81 (1H, d, J=8.4 Hz), 6.73-6.70 (2H, m), 2.42 (3H, s); ¹³C NMR (100 MHz, MeOD) δ 197.0, 161.3, 149.9, 147.3, 146.8, 141.7, 132.5, 131.6, 128.1, 124.0, 123.4, 119.2, 116.5, 115.4, 113.3, 21.26; LRMS (ESI) calcd. for C₁₆H₁₅O₄ [M+H]⁺: 271.10, found: 271.10; HRMS (ESI) calcd. for C₁₆H₁₅O₄ [M+H]⁺: 271.0965, found: 271.0958; M.P=208.2° C.

### Compound 7i: (E)-3-(3,4-dihydroxyphenyl)-1-(4-hydroxy-2-methoxyphenyl)prop-2-en-1-one

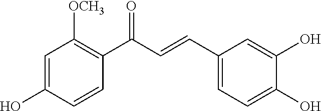 [SMILES: COc1cc(O)ccc1C(=O)/C=C/c1ccc(O)c(O)c1]

Compound 7i was isolated via Preparation B (yellow solid, 46% yield), and ¹H NMR, ¹³C NMR, LRMS, HRMS and MP data were measured as follows:

¹H NMR (400 MHz, MeOD) δ 7.59 (1H, d, J=8.8 Hz), 7.51 (1H, d, J=15.6 Hz), 7.38 (1H, d, J=15.2 Hz), 7.12 (1H, d, J=2.0 Hz), 7.00 (1H, dd, J=8.4, 2.0 Hz), 6.80 (1H, d, J=8.0 Hz), 6.53 (1H, d, J=2.0 Hz), 6.47 (1H, dd, J=8.4, 2.0 Hz), 3.91 (3H, s); ¹³C NMR (100 MHz, MeOD) δ 193.0, 164.5, 162.5, 149.5, 146.8, 144.5, 133.7, 128.6, 125.1, 123.3, 121.7, 116.5, 115.2, 108.9, 100.1, 56.15; LRMS (ESI) calcd. for C₁₆H₁₅O₅ [M+H]⁺: 287.09, found: 287.10; HRMS (ESI) calcd. for C₁₆H₁₅O₅ [M+H]⁺: 287.0914, found: 287.0906; M.P=200.4° C.

### Compound 7j: (E)-3-(3,4-dihydroxyphenyl)-1-(4-hydroxy-2-isopropoxyphenyl)prop-2-en-1-one

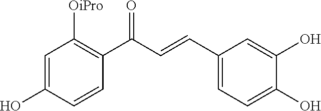

Compound 7j was isolated via Preparation A (yellow solid, 53% yield), and ¹H NMR, ¹³C NMR, LRMS, HRMS and MP data were measured as follows:

¹H NMR (400 MHz, MeOD) δ 7.58 (1H, d, J=8.8 Hz), 7.47 (2H, d, J=2.8 Hz), 7.11 (1H, d, J=2.0 Hz), 7.00 (1H, dd, J=8.4, 2.4 Hz), 6.81 (1H, d, J=8.0 Hz), 6.51 (1H, d, J=2.4 Hz), 6.45 (1H, dd, J=8.8, 2.4 Hz), 4.69 (1H, hept, 6.0 Hz), 1.40 (6H, d, J=6.0 Hz); ¹³C NMR (100 MHz, MeOD) δ 193.4, 160.6, 146.8, 143.9, 133.7, 128.7, 125.5, 123.1, 122.9, 116.5, 115.2, 109.1, 102.2, 72.21, 22.43; LRMS (ESI) calcd. for C₁₈H₁₉O₅ [M+H]⁺: 315.12, found: 315.20; HRMS (ESI) calcd. for C₁₈H₁₉O₅ [M+H]⁺: 315.1227, found: 315.1219; M.P=80.4° C.

### Compound 7k: (E)-3-(3,4-dihydroxyphenyl)-1-(2,4-dimethoxyphenyl)prop-2-en-1-one

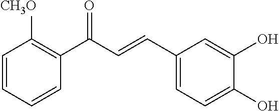

Compound 7k was isolated via Preparation A (yellow solid, 63% yield) and ¹H NMR, ¹³C NMR, LRMS, HRMS and MP data were measured as follows:

¹H NMR (400 MHz, MeOD) δ 7.65 (1H, d, J=8.4 Hz), 7.51 (1H, d, J=15.6 Hz), 7.34 (1H, d, J=15.6 Hz), 7.12 (1H, d, J=2.0 Hz), 7.00 (1H, dd, J=8.4, 2.0 Hz), 6.81 (1H, d, J=8.4 Hz), 6.65-6.61 (2H, m), 3.93 (3H, s), 3.89 (3H, s); ¹³C NMR (100 MHz, MeOD) δ 193.3, 166.0, 162.0, 149.7, 146.8, 145.0, 133.4, 128.5, 124.9, 123.4, 116.5, 115.2, 106.7, 99.53, 56.20; LRMS (ESI) calcd. for C₁₇H₁₇O₅ [M+H]⁺: 301.10, found: 301.10; HRMS (ESI) calcd. for C₁₇H₁₇O₅ [M+H]⁺: 301.1071, found: 301.1065; M.P=162.9° C.

### Compound 7l: (E)-3-(3,4-dihydroxyphenyl)-1-(p-tolyl)prop-2-en-1-one

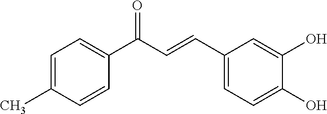 [SMILES: Cc1ccc(C(=O)/C=C/c2ccc(O)c(O)c2)cc1]

Compound 7l was isolated via Preparation A (yellow solid, 66% yield) and ¹H NMR, ¹³C NMR, LRMS, HRMS and MP data were measured as follows:

¹H NMR (400 MHz, MeOD) δ 7.96 (2H, d, J=8.0 Hz), 7.68 (1H, d, J=15.6 Hz), 7.50 (1H, d, J=15.2 Hz), 7.36 (2H, d, J=8.0 Hz), 7.20 (1H, d, J=2.0 Hz), 7.11 (1H, dd, J=8.4, 2.4 Hz), 6.83 (1H, d, J=8.4 Hz), 2.44 (3H, s); ¹³C NMR (100 MHz, MeOD) δ 192.2, 150.0, 147.0, 146.8, 145.0, 137.1, 130.4, 129.6, 128.3, 123.6, 119.6, 116.6, 115.7, 21.64; LRMS (ESI) calcd. for C₁₆H₁₅O₃ [M+H]⁺: 255.10, found: 255.10; HRMS (ESI) calcd. for C₁₆H₁₅O₃ [M+H]⁺: 255.1016, found: 255.1014; M.P=190.9° C.

### Compound 7m: (E)-1-(4-chlorophenyl)-3-(3,4-dihydroxyphenyl)prop-2-en-1-one

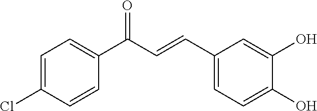 [SMILES: O=C(/C=C/c1ccc(O)c(O)c1)c1ccc(Cl)cc1]

Compound 7m was isolated via Preparation A (yellow solid, 63% yield), and ¹H NMR, ¹³C NMR, LRMS, HRMS and MP data were measured as follows:

¹H NMR (400 MHz, MeOD) δ 8.06 (2H, d, J=8.8 Hz), 7.71 (1H, d, J=15.6 Hz), 7.56 (2H, d, J=8.8 Hz), 7.49 (1H, d, J=15.6 Hz), 7.21 (1H, d, J=2.0 Hz), 7.14 (1H, dd, J=8.4, 2.4 Hz), 6.84 (1H, d, J=8.4 Hz); ¹³C NMR (100 MHz, MeOD) δ 191.1, 147.8, 146.9, 140.0, 138.2, 131.1, 129.9, 128.1, 123.8, 119.1, 116.6, 115.8; LRMS (ESI) calcd. for C₁₅H₁₂O₃Cl[M+H]⁺: 275.05, found: 275.10; HRMS (ESI) calcd. for C₁₅H₁₂O₃Cl [M+H]⁺: 275.0469, found: 275.0465; M.P=205.4° C.

### Compound 8a: (E)-4-(3-(2,4-diacetoxyphenyl)-3-oxoprop-1-en-1-yl)-1,2-phenylene diacetate

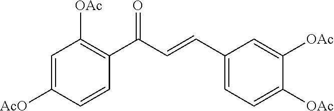 [SMILES: CC(=O)Oc1ccc(C(=O)/C=C/c2ccc(OC(C)=O)c(OC(C)=O)c2)c(OC(C)=O)c1]

The solution of (E)-1-(2,4-dihydroxyphenyl)-3-(3,4-dihydroxyphenyl)prop-2-en-1-one (compound 1, 500 mg, 1.8 mmol)) in Ac₂O (8 mL) was heated to 120° C.-130° C. and stirred at this temperature for 5 h. After cooling to room temperature, the resulting mixture was concentrated in a vacuum condition. From the residue, 397 mg of compound 8a was obtained by silica gel column chromatography (silica gel:n-hexane-ethylacetate=1:2) (white solid, 90% yield). ¹H NMR, ¹³C NMR, LRMS, HRMS and MP data were measured as follows:

¹H NMR (400 MHz, MeOD) b 7.86 (2H, d, J=8.4 Hz), 7.63-7.61 (2H, m), 7.58 (1H, d, J=16.0 Hz), 7.35 (1H, d, J=12.4 Hz), 7.31 (1H, d, J=5.2 Hz), 7.22 (1H, dd, J=8.4, 2.4 Hz), 7.10 (1H, d, J=2.4 Hz), 2.33 (3H, s), 2.31 (6H, d, J=3.2 Hz), 2.23 (3H, s); ¹³C NMR (100 MHz, MeOD) b 188.8, 168.6, 168.0, 153.4, 149.5, 143.7, 142.4, 142.3, 133.1, 131.2, 128.9, 127.5, 125.4, 124.1, 123.3, 119.6, 117.6, 20.76, 20.32; LRMS (ESI) calcd. for C₂₃H₂₁O₉ [M+H]⁺: 441.12, found: 441.10; HRMS (ESI) calcd. for C₂₃H₂₁O₉ [M+H]⁺: 441.1180, found: 441.1171; M.P=131.3° C.

### Compound 8b: (E)-4-(3-(2,4-bis(benzoyloxy)phenyl)-3-oxoprop-1-en-1-yl)-1,2-phenylene dibenzoate

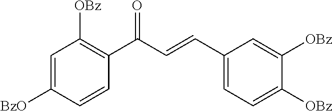

(E)-1-(2,4-dihydroxyphenyl)-3-(3,4-dihydroxyphenyl)prop-2-en-1-one (compound 1, 500 mg, 1.8 mmol) was dissolved in anhydrous pyridine (5 mL), treated with benzoylchloride (2.1 ml, 18.4 mmol), refluxed for 4 h, left at 22° C.-24° C. overnight, and diluted with cold H₂O the next day. The ethyl acetate layer was washed three times with water (30 ml×3) and dried using anhydrous MgSO₄.

After removing the solvent, 413 mg of compound 8b was obtained by silica gel column chromatography (silica gel: n-hexane-ethyl acetate=1:2) (white solid, 60% yield). ¹H NMR, ¹³C NMR, LRMS, HRMS and MP data were measured as follows:

¹H NMR (400 MHz, MeOD) b 8.24-8.21 (2H, m), 8.16-8.13 (2H, m), 8.05-7.99 (6H, m), 7.65-7.57 (7H, m), 7.51-7.37 (11H, m); ¹³C NMR (100 MHz, MeOD) b 188.6, 164.2, 163.4, 153.7, 149.7, 143.7, 142.4, 134.2, 133.4, 132.7, 131.1, 129.7, 129.6, 129.4, 129.2, 128.9, 128.4, 128.1, 127.8, 125.7, 124.1, 123.2, 120.1, 118.0; LRMS (ESI) calcd. for C₄₃H₂₉O₉ [M+H]⁺: 689.18, found: 689.20; HRMS (ESI) calcd. for C₄₃H₂₉O₉ [M+H]⁺: 689.1812, found: 689.1794; M.P=36.7° C.

### Compound 10: 1-(2-hydroxy-4-(methoxymethoxy)phenyl)ethanone

1-(2,4-dihydroxyphenyl)ethanone (compound 5a, 1.5 g, 10 mmol) in 12 mL acetone was added to K₂CO₃ (1.4 g, 10 mmol), and at room temperature, in an Ar atmospheric condition, chloro(methoxy)methane (885 mg, 11 mmol) was added thereto with slow stirring.

The reaction mixture was stirred for 4 h at room temperature and filtered. The solid was washed with EtOAc (80 mL) and the filtrate was washed with NaH₂PO₄ (sat. 50 mL) and water (80 mL), dried using MgSO₄ and concentrated.

From the crude product, 1.2 g of compound 10 was obtained by silica gel column chromatography (silica gel: n-hexane-ethyl acetate=1:4) (yellow white solid, 61% yield). ¹H NMR, ¹³C NMR, LRMS, HRMS and MP data were measured as follows:

¹H NMR (400 MHz, DMSO-d6) δ 12.49 (1H, s), 7.85 (1H, d, J=8.8 Hz) 6.60 (1H, dd, J=8.8, 2.4 Hz), 5.53 (1H, d, J=2.4 Hz), 5.27 (2H, s), 3.39 (3H, s), 2.57 (3H, s); ¹³C NMR (100 MHz, DMSO-d6) b 203.1, 163.5, 133.2, 114.5, 108.0, 102.9, 93.62, 55.90, 26.71; HRMS (ESI) calcd. for C₁₀H₁₂O₄ [M+H]⁺: 197.08, found: 197.10; HRMS (ESI) calcd. for C₁₀H₁₂O₄ [M+H]⁺: 197.0825, found: 197.0815; M.P=60.2° C.

### Compound 11: 3,4-bis(methoxymethoxy)benzaldehyde

Potassium carbonate (4.0 g, 19 mmol) and methoxymethyl chloride (2.3 g, 2.2 mL, 19 mmol) were added to a solution of 3,4-dihydroxybenzaldehyde (compound 6a, 1 g, 7.2 mmol) in acetone (40 mL). The mixture was refluxed for 2.5 hours, and the precipitate was filtered therefrom. The solvent was then removed under reduced pressure.

The residue was dissolved in CH₂C₁₂ (30 mL), washed with water (80 mL), dried using MgSO₄ and concentrated.

From the crude product, 1.1 g of compound 11 was obtained by chromatography on silica (n-hexane-ethyl acetate, 1:4) (white solid, 70% yield). ¹H NMR, ¹³C NMR, LRMS, HRMS and MP data were measured as follows:

¹H NMR (400 MHz, DMSO-d6) δ 9.85 (1H, s), 7.60-7.58 (2H, m), 7.31 (1H, d, J=8.0 Hz), 5.30 (4H, d, J=23.6 Hz), 3.42 (6H, d, J=2.0 Hz); ¹³C NMR (100 MHz, DMSO-d6) b 191.3, 152.1, 146.9, 130.5, 126.1, 115.6, 115.3, 94.67, 94.34, 55.97, 55.79; HRMS (ESI) calcd. for C₁₁H₁₄O₅ [M+H]⁺: 227.09, found: 227.10; HRMS (ESI) calcd. for C₁₁H₁₄O₅ [M+H]⁺: 227.0935, found: 227.0935; M.P=44.6° C.

### Compound 12: (E)-3-(3,4-bis(methoxymethoxy)phenyl)-1-(2-hydroxy-4-(methoxymethoxy)-phenyl)-prop-2-ene-1-one

1-(2-hydroxy-4-(methoxymethoxy)phenyl)-ethanone (compound 10, 1.0 g, 5.09 mmol) and 3,4-bis(methoxymethoxy)benzaldehyde (compound 11, 1.2 g, 5.09 mmol) were dissolved in ethanol (10 mL). 60% KOH (aq) (2 mL) was added thereto and the mixture was left at room temperature for 6 h. The solvent was removed therefrom under reduced pressure, and the residue was extracted three times with 30 ml of diethyl ether. The diethyl ether layer was washed three times with water (30 ml×3) and dried using anhydrous MgSO₄.

After removing the solvent, 1.3 g of compound 12 was obtained by silica gel column chromatography (silica gel: n-hexane-ethyl acetate=1:2) (yellow solid, 63% yield). ¹H NMR, ¹³C NMR, LRMS, HRMS and MP data were measured as follows:

¹H NMR (700 MHz, DMSO-d6) δ 13.36 (1H, s), 8.25 (1H, dd, J=9.1, 3.5 Hz), 7.88 (1H, dd, J=15.4, 3.5 Hz), 7.77 (1H, dd, J=15.4, 3.5 Hz), 7.76-7.68 (1H, m), 7.52-7.50 (1H, m), 7.18 (1H, dd, J=8.4, 4.2 Hz), 6.66-6.64 (1H, m), 6.59-6.58 (1H, m) 5.29 (3H, dd, J=13.3, 4.2 Hz) 3.45 (3H, d, J=4.2 Hz), 3.41 (6H, dd, J=7.7, 4.2 Hz); ¹³C NMR (175 MHz, DMSO-d6) b 133.0, 129.2, 125.2, 120.1, 117.7, 117.0, 115.2, 108.6, 103.6, 95.53, 95.12, 94.33, 56.51, 56.43, 56.37; HRMS (ESI) calcd. for C₂₁H₂₄O₈ [M+H]⁺: 405.16, found: 405.10; HRMS (ESI) calcd. for C₂₁H₂₄O₈ [M+H]⁺: 405.1582, found: 405.1592; M.P=107.8° C.

### Compound 13a: (E)-2-(3-(3,4-bis(methoxymethoxy)phenyl)acryloyl)-5-(methoxymethoxy)phenyl benzoate

(E)-3-(3,4-bis(methoxymethoxy)phenyl)-1-(2-hydroxy-4-(methoxymethoxy)phenyl)prop-2-en-1-one (Compound 12, 500 mg, 1.0 mmol) was dissolved in methylene chloride (10 mL). TEA (164 μL, 1.18 mmol) and benzoylchloride (137 μL, 1.18 mmol) were added and the mixture was left at room temperature for 6 hours. The solvent was removed under reduced pressure, and the residue was extracted three times with 20 ml of ethyl acetate. The ethyl acetate layer was washed three times with water (20 ml×3) and dried using anhydrous MgSO₄.

After removing the solvent, 430 mg of compound 13a was obtained by silica gel column chromatography (silica gel: n-hexane-ethyl acetate=1:2) (yellow solid, 86% yield). ¹H NMR, ¹³C NMR, LRMS, HRMS and MP data were measured as follows:

¹H NMR (700 MHz, DMSO-d6) δ 7.97 (2H, d, J=7.7 Hz), 7.92 (1H, d, J=9.1 Hz), 7.43 (1H, d, J=11.9 Hz), 7.35 (1H, d, J=14.7 Hz), 7.32 (1H, d, J=7.7 Hz), 7.25 (1H, d, J=8.4 Hz), 7.12 (1H, d, J=8.4 Hz), 7.08 (2H, d, J=9.1 Hz), 5.33 (2H, s), 5.23 (4H, d, J=19.6 Hz), 3.42 (9H, t, J=8.1 Hz), 2.38 (3H, s)¹³C NMR (175 MHz, DMSO-de) b 188.8, 164.8, 160.7, 150.9, 149.8, 147.2, 144.8, 143.8, 132.1, 130.3, 129.7, 129.5, 129.0, 126.4, 126.1, 124.4, 124.0, 117.2, 116.9, 114.1, 111.7, 95.49, 95.12, 94.52, 56.43, 56.36, 56.32, 21.63; HRMS (ESI) calcd. for C₂₉H₃₀O₉ [M+H]⁺: 523.20, found: 523.20; HRMS (ESI) calcd. for C₂₉H₃₀O₉ [M+H]⁺: 523.2006, found: 523.2006; M.P=47.2° C.

### Compound 14a: (E)-2-(3-(3,4-dihydroxyphenyl)acryloyl)-5-hydroxyphenyl benzoate

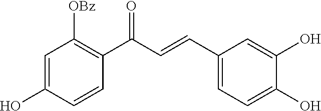

(E)-2-(3-(3,4-bis(methoxymethoxy)phenyl)acryloyl)-5-(methoxymethoxy)phenyl benzoate (compound 13a, 400 mg, 0.787 mmol) was dissolve in methanol (1.5 mL) and 0.25 mL of concentrated hydrochloric acid was added thereto. The mixture was then heated to 45° C. for 2 h. The solvent was removed under reduced pressure to obtain a solid residue, which was dissolved in ethyl acetate (10 mL) and washed with saturated NaHCO₃-saturated solution (3×20 mL). The organic phase was dried using MgSO₄.

After removing the solvent, 220 mg of compound 14a was obtained by silica gel column chromatography (silica gel: n-hexane-ethyl acetate=1:1) (yellow solid, 74% yield). ¹H NMR, ¹³C NMR, LRMS, HRMS and MP data were measured as follows:

¹H NMR (400 MHz, MeOD) b 8.12 (2H, dd, J=8.4, 1.6 Hz), 7.75 (1H, d, J=8.4 Hz), 7.65-7.60 (1H, m), 7.47 (2H, t, J=8.4 Hz), 7.42 (1H, d, J=15.6 Hz), 7.11 (1H, d, J=16.0 Hz), 6.89-6.85 (2H, m), 6.73-6.70 (2H, m); ¹³C NMR (100 MHz, MeOD) b 192.1, 166.5, 152.5, 149.8, 146.7, 134.9, 133.1, 131.2, 130.4, 129.7, 1.9, 125.2, 123.4, 123.0, 116.4, 115.5, 114.3, 111.4; LRMS (ESI) calcd. for C₂₂H₁₇O₆ [M+H]⁺: 377.10, found: 377.10; HRMS (ESI) calcd. for C₂₂H₁₇O₆ [M+H]⁺: 377.1020, found: 377.1007; M.P=204.9° C.

### Compound 14b: (E)-2-(3-(3,4-dihydroxyphenyl)acryloyl)-5-hydroxyphenyl 4-methylbenzoate

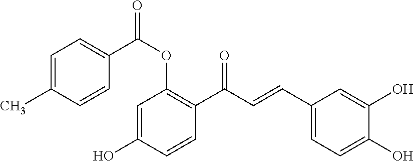 [SMILES: Cc1ccc(C(=O)Oc2cc(O)ccc2C(=O)/C=C/c2ccc(O)c(O)c2)cc1]

Compound 14b was isolated (yellow solid, 51% yield) and ¹H NMR, ¹³C NMR, LRMS, HRMS and MP data were measured as follows:

¹H NMR (400 MHz, MeOD) b 7.98 (2H, d, J=8.0 Hz), 7.74 (1H, d, J=15.6 Hz), 7.39 (1H, d, J=8.4 Hz), 7.26 (2H, d, J=7.6 Hz), 7.08 (1H, d, J=16.0 Hz), 6.92 (1H, d, J=2.0 Hz), 6.86-6.82 (2H, m), 6.71-6.68 (2H, m), 2.40 (3H, s); ¹³C NMR (100 MHz, MeOD) b 192.1, 166.6, 163.5, 152.6, 149.8, 146.6, 146.1, 133.1, 131.2, 130.3, 128.0, 127.5, 125.3, 123.4, 123.2, 116.3, 115.4, 114.3, 111.4, 21.72; LRMS (ESI) calcd. for C₂₃H₁₉O₆ [M+H]⁺: 391.12, found: 391.10; HRMS (ESI) calcd. for C₂₃H₁₉O₆ [M+H]⁺: 391.1176, found: 391.1171; M.P=185.7° C.

### Compound 14c: (E)-2-(3-(3,4-dihydroxyphenyl)acryloyl)-5-hydroxyphenyl 4-methoxybenzoate

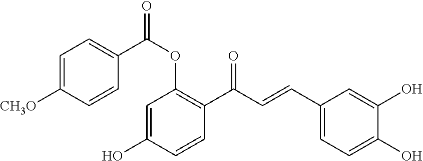

Compound 14c was isolated (brown solid, 44% yield) and ¹H NMR, ¹³C NMR, LRMS, HRMS and MP data were measured as follows:

¹H NMR (400 MHz, MeOD) b 8.05 (2H, d, J=8.8 Hz), 7.73 (1H, d, J=8.4 Hz), 7.39 (1H, d, J=15.6 Hz), 7.09 (1H, d, J=15.6 Hz), 6.95-6.93 (3H, m), 6.86-6.82 (2H, m), 6.71-6.69 (2H, m), 3.85 (3H, s); ¹³C NMR (100 MHz, MeOD) b 192.1, 166.2, 165.7, 163.5, 152.7, 149.8, 146.6, 146.4, 133.4, 133.1, 128.0, 125.5, 123.4, 123.3, 122.3, 116.3, 115.4, 115.0, 114.3, 111.4, 56.07; LRMS (ESI) calcd. for C₂₃H₁₉O₇ [M+H]⁺: 407.11, found: 407.10; HRMS (ESI) calcd. for C₂₃H₁₉O₇ [M+H]⁺: 407.1125, found: 407.1116; M.P=161.1° C.

### Compound 14d: (E)-2-(3-(3,4-dihydroxyphenyl)acryloyl)-5-hydroxyphenyl 4-methylbenzoate

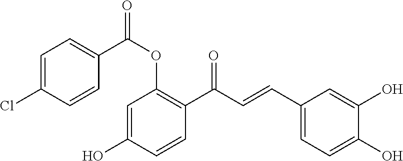 [SMILES: O=C(Oc1cc(O)ccc1C(=O)/C=C/c1ccc(O)c(O)c1)c1ccc(Cl)cc1]

Compound 14d was isolated (yellow solid, 61% yield) and ¹H NMR, ¹³C NMR, LRMS, HRMS and MP data were measured as follows:

¹H NMR (400 MHz, MeOD) b 8.08 (2H, d, J=8.4 Hz), 7.76 (1H, d, J=8.8 Hz), 7.47 (2H, d, J=8.8 Hz), 7.40 (1H, d, J=16.0 Hz), 7.09 (1H, d, J=16.0 Hz), 6.96 (1H, d, J=2.4 Hz), 6.88-6.85 (2H, m), 6.73-6.71 (2H, m); ¹³C NMR (100 MHz, MeOD) b 191.9, 163.6, 152.5, 149.9, 146.7, 146.6, 133.2, 130.0, 129.1, 127.9, 125.0, 123.4, 123.0, 116.4, 115.4, 114.4, 111.4; LRMS (ESI) calcd. for C₂₂H₁₆O₆Cl[M+H]⁺: 411.06, found: 411.10; HRMS (ESI) calcd. for C₂₂H₁₆O₆Cl [M+H]⁺: 411.0630, found: 411.0620; M.P=102.8° C.

### EXPERIMENTAL EXAMPLES

Compound 1 is butein and the therapeutic effect on lymphedema thereof is disclosed in Korean Patent Publication No. 10-1533197 (published on Jul. 2, 2015). However, compound 1 has poor physiological and chemical properties such as water solubility (79 μM, 21 μg/mL) and partition coefficient (log P, 0.42), and also, when treated in vitro at the concentration of 20 μM, IC₅₀ (43 μM) and efficacy (10% inhibition) were poor. This indicates that compound 1 is not the most suitable active ingredient for the treatment of lymphedema.

Accordingly, the inventors of the present application have studied to develop, through structural modification, a compound of which physicochemical properties (solubility, log P), and pharmacokinetic parameters, such as maximum concentration (Cmax) of the drug after oral administration, bioavailability (BA), half-life (T_{1/2}), total concentration (AUC), etc., are improved.

Anti-Inflammatory Effect

To isolate peritoneal macrophages, male Balb/c mice were intraperitoneally injected with 2 ml of 3.5% sterile thioglycolate (B D, Sparks, Md., USA) 4 days before being sacrificed. Mice were sacrificed by cervical dislocation, and peritoneal secretory cells were aseptically isolated through peritoneal washing with cold DMEM (HyClone, Logan, Utah, USA) containing 10% FBS (fetal bovine serum; HyClone) and 1% penicillin-streptomycin. After centrifugation, cells were resuspended and counted using a TC20 Cell Counter (Bio-Rad Laboratories, Hercules, Calif., USA). Peritoneal secretory cells were left in a 24-well plate overnight at 37° C., and non-adherent cells were removed. The cells were then stimulated with 100 ng/ml lipopolysaccharide (LPS) for 24 hours. The supernatant was collected during cytokine analysis, and the level of TNFα in the supernatant was measured using BD OptEIA mouse ELISA sets (BD Biosciences, San Diego, Calif., USA) according to the manufacturer's protocol.

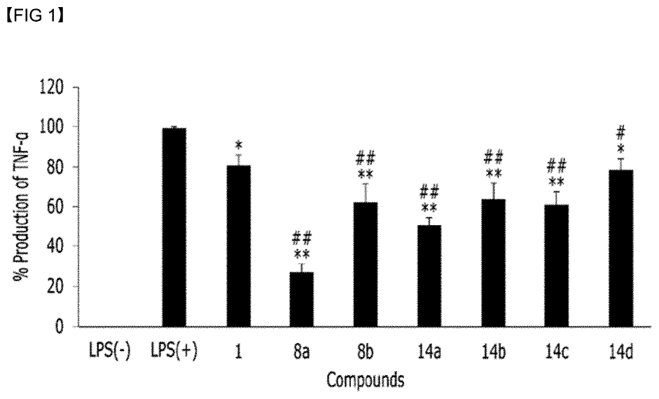

The measurements of the LPS-induced TNFα production level in mouse peritoneal macrophages exposed to the compound having a concentration of 20 μM are shown in FIG. 1. Data are mean±standard deviation of values for three mice, respectively (with respect to LPS, *: P<0.01, **: P<0.001; with respect to compound 1 #: P<0.01, ##: P<0.001). Referring to FIG. 1, it can be seen that all of the compounds 14a to 14d, 8a and 8b treatment groups have significantly better TNFα production inhibitory ability than the positive control butein treatment group.

In addition, in order to confirm the anti-inflammatory activity of the compound, the IC₅₀ values in relation to the dose-dependent inhibition level of TNFα production in macrophages are shown in Table 1 below. IC₅₀ (μM) values were measured twice, respectively, and expressed as mean±standard deviation. As can be seen in Table 1 below, the IC₅₀ value of the compound 7h treatment group was similar to that of the positive control butein treatment group, and compounds 7i, 7l, 7m, 14a to 14d, and 8a all showed significantly superior IC₅₀ values compared to butein.

TABLE 1

| | IC₅₀(μM) |
|---|---|
| compound 1 | 43.3 ± 1.72 |
| compound 7h | 48.4 ± 2.35 |
| compound 7i | 32.2 ± 2.15 |
| compound 7l | 18.6 ± 1.50 |
| compound 7m | 17.3 ± 2.11 |
| compound 14a | 14.6 ± 1.56 |
| compound 14b | 18.9 ± 1.15 |
| compound 14c | 20.8 ± 1.62 |
| compound 14d | 17.9 ± 1.84 |
| compound 8a | 12.8 ± 1.24 |

Physicochemical and Pharmacokinetic Parameters

Compounds were administered to 5-week-old male CD-1 mice through 1 mg/kg tail vein administration (administration volume of 5 mL/kg), 20 mg/kg oral administration, and 100 mg/kg oral administration, respectively. Three mice were used for each independent experiment. Blood samples were taken in an amount of 20 μL to 30 μL via the saphenous vein at 5, 15, 30, 60, 120, 180, 360, and 1440 minutes.

Table 2 below shows the physicochemical properties of representative compounds confirmed by parallel artificial membrane permeability assay (PAMPA). As can be seen in Table 2 below, it was confirmed that in terms of the solubility and permeability, the compound of the disclosure exhibited better properties compared than Compound 1.

TABLE 2

| | pKa | logP | Permeability | Solubility |
|---|---|---|---|---|
| compound 1 | 7.96/11.65 | 0.42 | −5.35 | 21 μg/mL |
| compound 7m | 9.23/11.58 | 3.77 | −4.33 | 28 μg/mL |
| compound 14a | 3.38/8.63/9.55 | 3.47 | −5.67 | 136 μg/mL |

In addition, pharmacokinetic parameters were calculated from non-compartmental analysis using Win Non-lin. Table 3 below shows the results of pharmacokinetic analysis of compounds 1 and 14a.

TABLE 3

| | Route of | Dosage | | | Cmax | AUC∞ | |
|---|---|---|---|---|---|---|---|
| Com- | adminis- | (mg/ | T_{1/2} | Tmax | (ng/ | (hr*ng/ | BA |
| pound | tration | Kg) | (hr) | (hr) | mL) | mL) | (%) |
| com- | I.V | 1 | — | 0.1 | 41 | 6.7 | — |
| pound | P.O | 20 | — | 0.3 | 43 | 21 | 15 |
| 1 | P.O | 100 | — | 0.3 | 601 | 251 | 37 |
| com- | I.V | 1 | 1.36 | 0.2 | 110 | 163 | — |
| pound | P.O | 20 | 0.57 | 0.4 | 83 | 141 | 4 |
| 14a | P.O | 100 | 0.35 | 0.4 | 1600 | 1224 | 7.5 |

In Table 3, T_{1/2} denotes the terminal half-life, Cmax denotes the maximum plasma concentration, AUC-O denotes the area under the plasma concentration-time curve from time zero to infinity, and indicates exposure concentration of whole blood, and BA represents bioavailability expressed as (AUCpo/AUCiv)×100. Compound 1 had too short terminal half-life and measurements thereof are two low, and thus T_{1/2} could not be measured. Cmax and AUC∞ are mean±SD values obtained from three male ICR mice. Regarding blood exposure during systemic circulation of the compound, the compound was orally administered at an amount of 100 mg/kg, and then, AUC (area under the curve) was measured, and the obtained measurements were compared.

Pharmacokinetic analysis through oral administration showed that compared to the case where unmodified compound 1 (AUC 251 h*ng/mL) was used, the case where compound 1 was delivered to the systemic circulation using compound 14a (AUC 1,224 h*ng/m) was significantly more effective.

Prophylactic Effect on Lymphedema Formation

Compounds 1, 7m, and 14a were each orally administered to mice for 7 days before surgical induction of lymphedema in mice.

The normal group did not undergo surgery and did not induce lymphedema. On the other hand, in the control group, the compound 1 treatment group, the compound 7m treatment group, and the compound 14a treatment group, lymphedema was induced through surgery. After surgery, the compounds were administered once every two days.

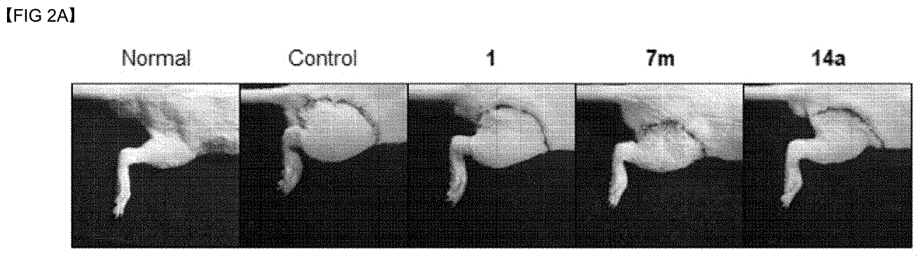

As can be seen in FIG. 2A, it was observed that the expansion of lymphedema in the right hind leg of the mouse was significantly suppressed. The volume of lymphedema was measured on the 7^{th} day after surgery, and the size of the lymphedema was recorded using a caliper ruler by a person who was blinded to the treatment status of the mice.

As can be seen in FIG. 2B, the volume of lymphedema on the 7^{th} day after surgery in the compound 1 treatment group, the compound 7m treatment group, and the compound 14a treatment group was reduced by 52%, 53%, and 70%, respectively, (with respect to the control group, *: P<0.005; with respect to compound 1, #: P<0.001).

Accordingly, it was confirmed that the modification of the pharmacokinetic properties of compound 14a ultimately significantly increased the prophylactic effect of lymphedema.

Histological and Immunofluorescence Analysis

Tissue samples were collected on the 7^{th} day after surgery to induce lymphedema at a location 15 mm-25 mm from the right leg of the mouse. The harvested tissue samples were fixed with 10% formalin solution, and the specimens were cut to a thickness of 5 μm. Sections were deparaffinized in 100% xylene, rehydrated with a series of 100% ethanol, 95% ethanol, 90% ethanol, 80% ethanol, and 70% ethanol, and washed with water. Sections were reacted with hematoxylin solution at room temperature. After washing in PBS, sections were stained with eosin at room temperature. Images were acquired by a Pannoramic viewer microscope (3DHISTECH Ltd., Hungary). Prepared slides were incubated using anti-mouse PPARγ (Santa Cruz Biotechnology, USA) and Alexa Fluor 488 and DAPI (Life Technologies, USA). Samples were analyzed using a Nikon ECLIPSE 80i microscope (Nikon Instruments Inc., Japan).

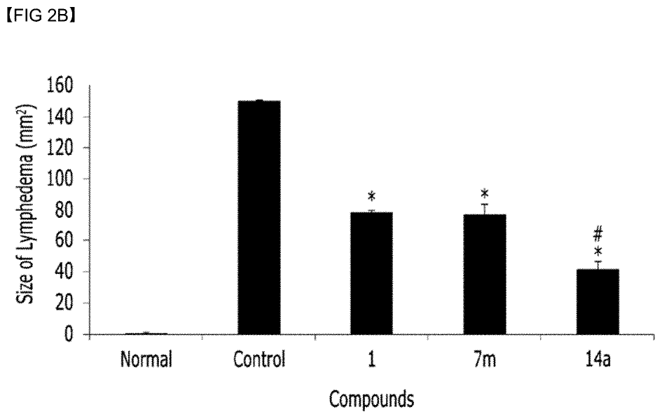

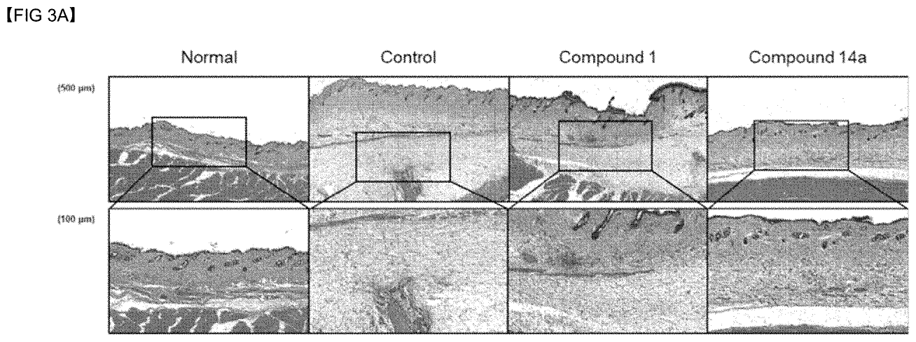

FIG. 3A shows H&E images of the right hind legs of mice taken through histological and immunofluorescence analysis in the normal group, the control group (lymphedema-inducing carrier injection group), the compound 1 treated group, and the compound 14a treated group. In FIG. 3A, the scale bar of the upper panel is 500 μm, and the scale bar of the lower panel is 100 μm. As can be seen in FIG. 3A, it can be seen that compound 14a decreased neutrophil infiltration and ameliorated the epidermal layer of lymphedema tissue.

In addition, the result of analyzing the mRNA expression of TNFα in lymphedema tissue is shown in FIG. 3B. As a result, it was confirmed that the expression level of TNFα mRNA was decreased in the compound 1 treatment group and the compound 14a treatment group (with respect to the control group, *: P<0.05; with respect to compound 1, #: P<0.05).

Next, the modulating effects of compound 1 and compound 14a on adipogenesis observed in lymphedema were analyzed.

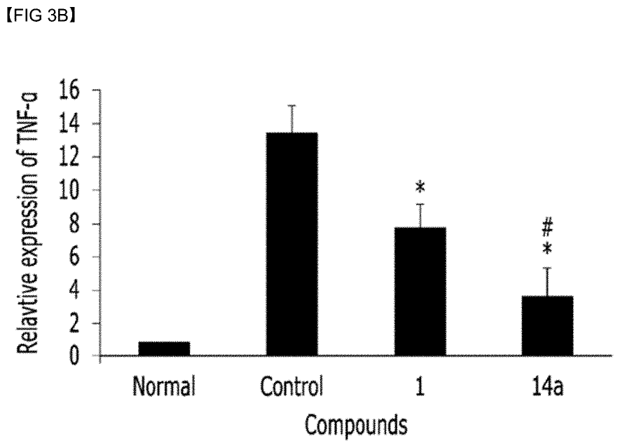

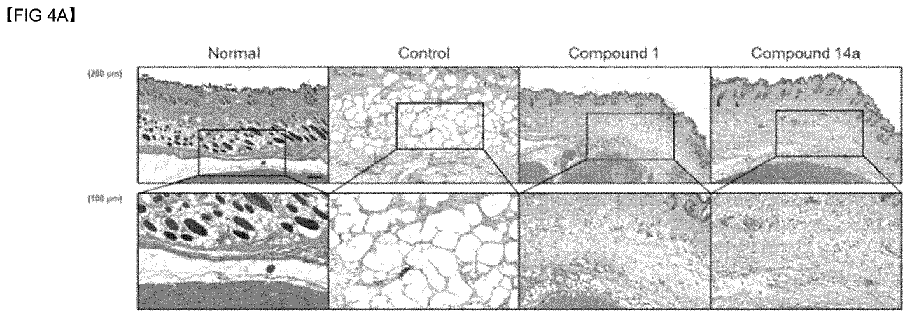

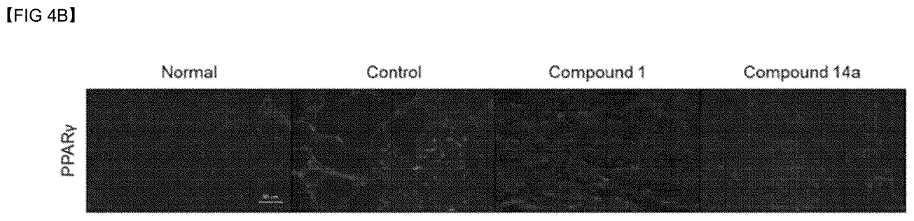

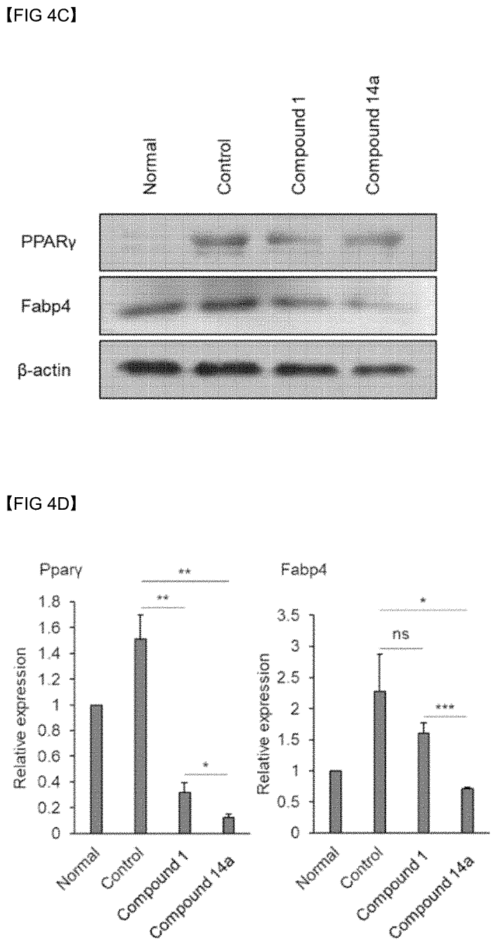

FIG. 4A shows H&E images of the right hind legs of mice taken through histological and immunofluorescence analysis in the normal group, the control group (lymphedema-inducing carrier injection group), the compound 1 treated group, and the compound 14a treated group. In FIG. 4A, the scale bar of the upper panel is 200 μm, and the scale bar of the lower panel is 100 μm. As can be seen in FIG. 4A, it was confirmed that compound 1 and compound 14a had the prophylactic effect on lymphedema tissue by inhibiting the development of adipose tissue in the epidermal layer.

In addition, peroxisome activated receptor γ (PPARγ) is a major regulator of lipogenesis, and as shown in FIG. 4B showing the results of immunofluorescence staining, the distribution of PPARγ in the epidermal layer of lymphedema tissue after treatment with compound 1 and compound 14a was suppressed.

As can be seen in FIG. 4C showing a western blot image, it was confirmed that compound 1 and compound 14a reduced the expression of PPARγ, which is a lipogenic biomarker, and a target protein Fabp4 (fatty acid binding protein 4) thereof in lymphedema tissue. In particular, in FIG. 4D showing the levels of PPARγ and Fabp4 mRNA, it can be seen that compound 1 exhibits superior efficacy compared to the control group, but compound 14a exhibits more significant efficacy even compared to compound 1 (* P<0.05, ** P<0.01, *** P<0.001).

These two histological tests show that inflammation and lipogenesis affect the regulation of the pathophysiological properties of lymphedema, and compound 14a significantly regulates lymphedema through the anti-inflammatory and anti-lipogenic effects compared to the control group and even to compound 1.

What is claimed is:

1. A compound of Formula 1 or Formula 2, or a pharmaceutically, food, or cosmetically acceptable salt thereof: 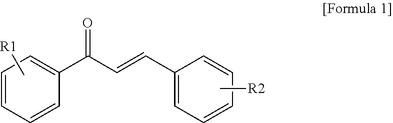
wherein, in this formula,
R1 and R2 are each independently one or more selected from a group consisting of —H, —OH, —OR3, —OCOR3, —NO₂, —CN, —SH, —SR3, —COR4, —COOR4, —CONHR4, —CON(R4)₂, —NH₂, —NHR4, —N(R4)₂, —NHCOR4, —N(R5)COR₄, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R3 are each independently selected from the group consisting of a C₁-C₆ alkyl group, a C₃-C₁₀ aryl group, 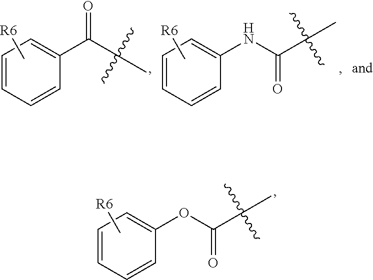
R6 are each independently one or more selected from the group consisting of —H, —OH, —OR7, —NO₂, —CN, a C₁-C₆ alkyl group, a C₁-C₆ alkoxy group, halogen, —NH₂, —NHR7, and —N(R7)₂,
R7 is selected from the group consisting of a C₁-C₆ alkyl group, an allyl group, and a heteroallyl group,
R4 is selected from the group consisting of a C₁-C₆ alkyl group, an allyl group, and a heteroallyl group,
R5 is selected from the group consisting of a C₁-C₆ alkyl group, an allyl group, and a heteroallyl group,
provided that butein is excluded from the compound of Formula 1; 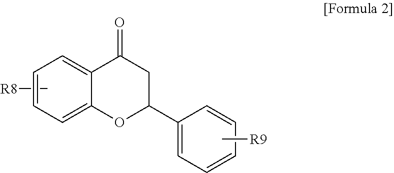
wherein, in this formula,
R8 and R9 are each independently one or more selected from the group consisting of —H, —OH, —OR10, —NO₂, —CN, —SH, —SR10, —COR11, —COOR11, —CONHR11, —CON(R11)₂, —NH₂, —NHR11, —N(R11)₂, —NHCOR11, —N(R12)COR11, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R10 is each independently selected from the group consisting of a C₁-C₆ alkyl group, a C₃-C₁₀ aryl group, 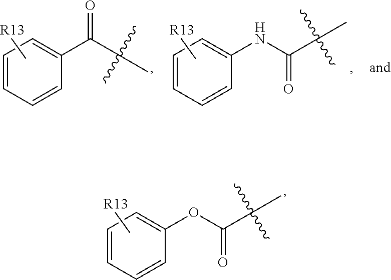
R13 is each independently one or more selected from the group consisting of —H, —OH, —NO₂, —CN, a C₁-C₆ alkyl group, a C₁-C₆ alkoxy group, halogen, —NH₂, —NHR14, and —N(R14)₂,
R14 is selected from the group consisting of a C₁-C₆ alkyl group, an allyl group, and a heteroallyl group,
R11 is selected from the group consisting of a C₁-C₆ alkyl group, an allyl group, and a heteroallyl group, and
R12 is selected from the group consisting of a C₁-C₆ alkyl group, an allyl group, and a heteroallyl group.

2. The compound or pharmaceutically, food, or cosmetically acceptable salt thereof of claim 1, wherein
the hetero atom is at least one compound selected from the group consisting of O, N and S.

3. The compound or pharmaceutically, food, or cosmetically acceptable salt thereof of claim 1, wherein
the compound or the pharmaceutically, food, or cosmetically acceptable salt thereof is selected from the group consisting of:
(E)-1-(2,4-dihydroxyphenyl)-3-(4-hydroxy-3-methoxyphenyl)prop-2-en-1-one;
(E)-1-(2,4-dihydroxyphenyl)-3-(3-hydroxy-4-methoxyphenyl)prop-2-en-1-one;
(E)-1-(2,4-dihydroxyphenyl)-3-(3-fluoro-4-hydroxyphenyl)prop-2-en-1-one;
(E)-1-(2,4-dihydroxyphenyl)-3-(4-fluoro-3-hydroxyphenyl)prop-2-en-1-one;
(E)-1-(2,4-dihydroxyphenyl)-3-(4-hydroxyphenyl)prop-2-en-1-one;
(E)-1-(2,4-dihydroxyphenyl)-3-(3-hydroxyphenyl)prop-2-en-1-one;
(E)-3-(3,4-dihydroxyphenyl)-1-(2-fluoro-4-hydroxyphenyl)prop-2-en-1-one;
(E)-3-(3,4-dihydroxyphenyl)-1-(4-hydroxy-2-methylphenyl)prop-2-en-1-one;
(E)-3-(3,4-dihydroxyphenyl)-1-(4-hydroxy-2-methoxyphenyl)prop-2-en-1-one;
(E)-3-(3,4-dihydroxyphenyl)-1-(4-hydroxy-2-isopropoxyphenyl)prop-2-en-1-one;
(E)-3-(3,4-dihydroxyphenyl)-1-(2,4-dimethoxyphenyl)prop-2-en-1-one;
(E)-3-(3,4-dihydroxyphenyl)-1-(p-tolyl)prop-2-en-1-one;
(E)-1-(4-chlorophenyl)-3-(3,4-dihydroxyphenyl)prop-2-en-1-one;
(E)-4-(3-(2,4-diacetoxyphenyl)-3-oxoprop-1-en-1-yl)-1,2-phenylene diacetate;
(E)-4-(3-(2,4-bis(benzoyloxy)phenyl)-3-oxoprop-1-en-1-yl)-1,2-phenylene dibenzoate;
(E)-2-(3-(3,4-dihydroxyphenyl)acryloyl)-5-hydroxyphenyl benzoate;
(E)-2-(3-(3,4-dihydroxyphenyl)acryloyl)-5-hydroxyphenyl 4-methylbenzoate;
(E)-2-(3-(3,4-dihydroxyphenyl)acryloyl)-5-hydroxyphenyl 4-methoxybenzoate, and
(E)-2-(3-(3,4-dihydroxyphenyl)acryloyl)-5-hydroxyphenyl 4-chlorobenzoate.

4. The compound or pharmaceutically, food, or cosmetically acceptable salt thereof of claim 1, wherein
the compound or pharmaceutically, food, or cosmetically acceptable salt thereof is for treatment, prevention, or amelioration of lymphedema.

5. A pharmaceutical composition for treating or preventing lymphedema, comprising a compound of Formula 1 or Formula 2, or pharmaceutically acceptable salt thereof: 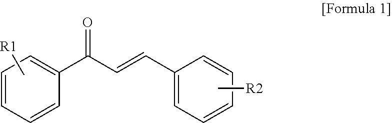
wherein, in this formula,
R1 and R2 are each independently one or more selected from the group consisting of —H, —OH, —OR3, —OCOR3, —NO₂, —CN, —SH, —SR3, —COR4, —COOR4, —CONHR4, —CON(R4)₂, —NH₂, —NHR4, —N(R4)₂, —NHCOR4, —N(R5)COR₄, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R3 are each independently selected from the group consisting of a C₁-C₆alkyl group, a C₃-C₁₀ aryl group, 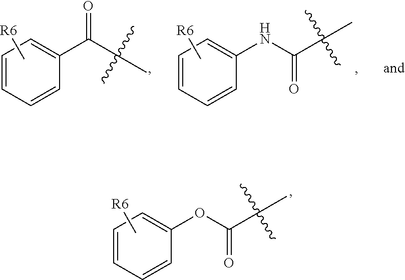
R6 are each independently one or more selected from the group consisting of —H, —OH, —OR7, —NO₂, —CN, a C₁-C₆ alkyl group, a C₁-C₆ alkoxy group, halogen, —NH₂, —NHR7, and —N(R7)₂,
R7 is selected from the group consisting of a C₁-C₆ alkyl group, an allyl group, and a heteroallyl group,
R4 is selected from the group consisting of a C₁-C₆ alkyl group, an allyl group, and a heteroallyl group,
R5 is selected from the group consisting of a C₁-C₆ alkyl group, an allyl group, and a heteroallyl group,
provided that butein is excluded from the compound of Formula 1;
[Formula 2] 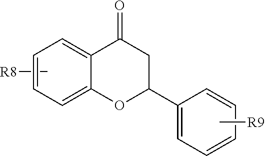
wherein, in this formula,
R8 and R9 are each independently one or more selected from the group consisting of —H, —OH, —OR10, —NO₂, —CN, —SH, —SR10, —COR11, —COOR11, —CONHR11, —CON(R11)₂, —NH₂, —NHR11, —N(R11)₂, —NHCOR₁₁, —N(R12)COR11, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R10 is each independently selected from the group consisting of a C₁-C₆ alkyl group, a C₃-C₁₀ aryl group, 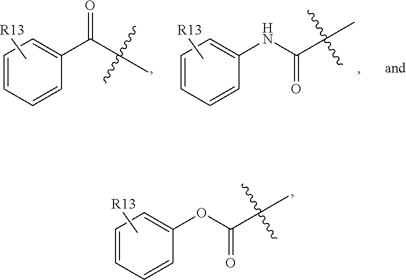
R13 is each independently one or more selected from the group consisting of —H, —OH, —NO₂, —CN, a C₁-C₆ alkyl group, a C₁-C₆ alkoxy group, halogen, —NH₂, —NHR14, and —N(R14)₂,
R14 is selected from the group consisting of a C₁-C₆ alkyl group, an allyl group, and a heteroallyl group,
R11 is selected from the group consisting of a C₁-C₆ alkyl group, an allyl group, and a heteroallyl group, and
R12 is selected from the group consisting of a C₁-C₆ alkyl group, an allyl group, and a heteroallyl group.

6. A method of treating or preventing lymphedema comprising administering to a subject a compound of Formula 1 or Formula 2, or a pharmaceutically acceptable salt thereof: 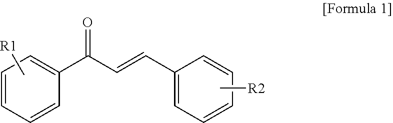
wherein, in this formula,
R1 and R2 are each independently one or more selected from the group consisting of —H, —OH, —OR3, —OCOR3, —NO₂, —CN, —SH, —SR3, —COR4, —COOR4, —CONHR4, —CON(R4)₂, —NH₂, —NHR4, —N(R4)₂, —NHCOR4, —N(R5)COR₄, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R3 are each independently selected from the group consisting of a C₁-C₆ alkyl group, a C₃-C₁₀ aryl group, 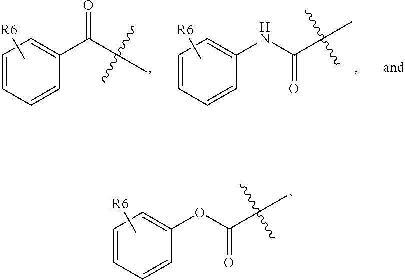
R6 are each independently one or more selected from the group consisting of —H, —OH, —OR7, —NO₂, —CN, a C₁-C₆ alkyl group, a C₁-C₆ alkoxy group, halogen, —NH₂, —NHR7, and —N(R7)₂,
R7 is selected from the group consisting of a C₁-C₆ alkyl group, an allyl group, and a heteroallyl group,
R4 is selected from the group consisting of a C₁-C₆ alkyl group, an allyl group, and a heteroallyl group,
R5 is selected from the group consisting of a C₁-C₆ alkyl group, an allyl group, and a heteroallyl group,
provided that butein is excluded from the compound of Formula 1; 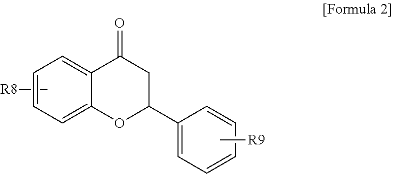
wherein, in this formula,
R8 and R9 are each independently one or more selected from the group consisting of —H, —OH, —OR10, —NO₂, —CN, —SH, —SR10, —COR11, —COOR11, —CONHR11, —CON(R11)₂, —NH₂, —NHR11, —N(R11)₂, —NHCOR₁₁, —N(R12)COR11, a C₁-C₆ alkyl group, a C₂-C₆ alkenyl group, a C₂-C₆ alkynyl group, halogen, an allyloxy group, a C₃-C₇ cycloalkyl group, an allyl group, a heteroallyl group having at least one hetero atom and consisting of 3-7 atoms, and a heterocycloalkyl group having at least one hetero atom and consisting of 3-7 atoms,
R10 is each independently selected from the group consisting of a C₁-C₆ alkyl group, a C₃-C₁₀ aryl group, 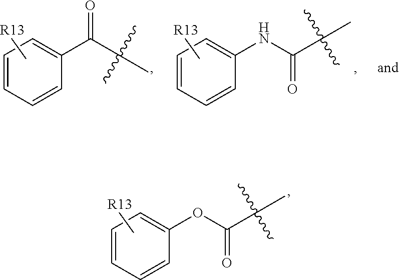
R13 is each independently one or more selected from the group consisting of —H, —OH, —NO₂, —CN, a C₁-C₆ alkyl group, a C₁-C₆ alkoxy group, halogen, —NH₂, —NHR14, and —N(R14)₂,
R14 is selected from the group consisting of a C₁-C₆ alkyl group, an allyl group, and a heteroallyl group,
R11 is selected from the group consisting of a C₁-C₆ alkyl group, an allyl group, and a heteroallyl group, and
R12 is selected from the group consisting of a C₁-C₆ alkyl group, an allyl group, and a heteroallyl group.
